(19) United States
(12) Patent Application Publication
(10) Pub. No.: US 2025/0186657 A1
(43) Pub. Date: Jun. 12, 2025

(54) NEURONAL REPLACEMENT AND REESTABLISHMENT OF AXONAL CONNECTIONS

(71) Applicant: THE TRUSTEES OF THE UNIVERISTY OF PENNSYLVANIA, Philadelphia, PA (US)

(72) Inventors: D. Kacy Cullen, Media, PA (US); Douglas H. Smith, Boothwyn, PA (US)

(21) Appl. No.: 18/952,880

(22) Filed: Nov. 19, 2024

Related U.S. Application Data
(63) Continuation of application No. 15/032,677, filed on Apr. 28, 2016, now abandoned.
(60) Provisional application No. 61/899,517, filed on Nov. 4, 2013.

(51) Int. Cl. A61L 27/52; A61K 35/30; A61L 27/20; A61L 27/22; A61L 27/24; A61L 27/26; A61L 27/34; A61L 27/36; A61L 27/38; A61L 27/54; A61L 27/58; C12N 5/0793

(52) U.S. Cl. CPC A61L 27/52; A61K 35/30; A61L 27/20; A61L 27/225; A61L 27/24; A61L 27/26; A61L 27/34; A61L 27/3604; A61L 27/3675; A61L 27/3878; A61L 27/54; A61L 27/58; C12N 5/0619; A61L 2300/412; A61L 2300/604; A61L 2300/606; A61L 2300/64; A61L 2400/06; A61L 2420/00; A61L 2430/32; C12N 2533/52; C12N 2533/56; C12N 2533/76; C12N 2533/78; C12N 2533/80; C12N 2533/90

(57) ABSTRACT

The present invention provides compositions and methods for modulation of neuronal networks in the CNS and/or PNS. In certain embodiments, the invention includes modulation of existing networks or restoring one or more damaged or lost axonal connections. In one embodiment, the invention comprises a tissue-engineered composition comprising an elongated tubular construct having at least one neuron and axon extending through the core of the construct.

### CROSS-REFERENCE TO RELATED APPLICATION

This application is a continuation of, and claims priority to, U.S. application Ser. No. 15/032,677 filed Apr. 28, 2016, which is a 35 U.S.C. § 371 national phase application from, and claims priority to, International Application No. PCT/US2014/063720 filed Nov. 3, 2014, and published under PCT Article 21 (2) in English, which claims priority under 35 U.S.C. § 119 (e) to U.S. Provisional Application No. 61/899,517 filed Nov. 4, 2013, all of which applications are incorporated herein by reference in their entireties.

### STATEMENT REGARDING FEDERALLY SPONSORED RESEARCH OR DEVELOPMENT

This invention was made with government support under grant number W81XWH-10-1-0941 and W81XWH-08-2-0034, awarded by the Army/Medical Research and Material Command and grant numbers NS048949, NS038104, NS056202 and NS043126, awarded by the National Institutes of Health. The government has certain rights in the invention.

### BACKGROUND OF THE INVENTION

The exquisite capabilities of the human brain rely on a multitude of long-distance connections between different specialized regions to enable profound parallel processing. These axon pathways frequently disconnect as a result of traumatic injury, stroke, and neurodegenerative diseases. Unfortunately, inhibition of axon growth, absence of directed guidance, and extreme distances to targets generally prevent functional regeneration (Belal and Ylikoski, 1983, J Laryngol Otol, 97 (1): 1-10; Cheng et al., 2010, Ann Neurol, 67 (6): 715-725; Curinga and Smith, 2008, Exp Neurol, 209 (2): 333-342; Huebner and Strittmatter, 2009, Results Probl Cell Differ 48:339-351; Levin et al., 1983, Am J Ophthalmol, 95 (3): 295-306; Marshall et al., 1988, Radiology, 167 (2): 517-522; Tallantyre et al., 2010, Mult Scler, 16 (4): 406-411). Several strategy thrusts for neuroregeneration, including traditional cell replacement as well as approaches to enhance axonal regeneration by modifying the extrinsic environment and/or augmenting the intrinsic ability of axons to regenerate, have only been partially successful in replacing the long-distance connections crucial for neural systems function.

For example, Denham et al. (2012, Front Cell Neurosci 6:11) implanted a single cell suspension of neurospheres into neonatal rats to determine the capacity of a human embryonic stem cell line to survive and integrate in vivo. They found that their grafts grew considerably and extended long-distance processes along existing white matter tracts (Denham et al., 2012, Front Cell Neurosci 6:11). In efforts to modify the extrinsic environment, Filous et al. (2010, Dev Neurobiol, 70 (12): 826-841) injected immature astrocytes, along with chondroitinase ABC, into rats with micro-lesions. They found that this dual treatment both reduced the presence of inhibitory ECM molecules, as well as provided a bridge for regenerating axons across the lesion (Filous et al., 2010, Dev Neurobiol, 70 (12): 826-841). Borisoff et al. (2003, Mol Cell Neurosci, 22 (3): 405-416) enhanced the intrinsic ability of axons to grow across inhibitory substrates by adding a Rho-kinase inhibitor to their cultures. In response, growth cone morphology altered distinctly to adopt features of growth cones on non-inhibitory substrates (Borisoff et al., 2003, Mol Cell Neurosci, 22 (3): 405-416). While these previous studies have replaced lost neurons, seen healthy axonal outgrowth, and reduced the presence of inhibitory molecules, none of these methods combine long-distance regeneration in a permissive environment with the specificity of directly guiding axons to appropriate targets.

Thus, there is a need in the art for improved compositions and methods for the restoration of long-distance axonal connections. The present invention satisfies this unmet need.

### BRIEF SUMMARY OF THE INVENTION

The present invention relates to a composition for modulating the activity of neurological network comprising: an elongated tubular construct having a first end and a second end, the construct comprising a tubular body having an outer surface and an inner surface defining a luminal core; and at least one axon extending through at least a portion of the core. In certain embodiments, the tubular body is a hydrogel. The hydrogel comprises at least one biopolymer, wherein the at least one biopolymer is at least one selected from the group consisting of hyaluronan, chitosan, alginate, collagen, dextran, pectin, carrageenan, polylysine, gelatin and agarose.

In another embodiment, the luminal core comprises at least one extracellular matrix protein. The at least one extracellular matrix protein is at least one selected from the group consisting of collagen, fibronectin, fibrin, hyaluronic acid, elastin, and laminin.

In yet another embodiment, the construct has an outer diameter from about 500 μm to about 1 mm and an inner diameter from about 125 μm to about 500 μm. The length of the construct is from about 0.1 mm to about 10 cm.

In yet another embodiment, the composition comprises at least one axon extending uni-directionally through the core. Alternatively, the composition comprises at least one axon extending bi-directionally through the core. In some instances, wherein the construct comprises a coating on the outer surface of the tubular body, and the coating provides hydration dependent mechanical properties to the construct. In one instance, the coating allows for needle-less delivery of the composition, and comprises carboxymethyl cellulose (CMC).

In yet another embodiment, the axon is of a neuron selected from the group consisting of a peripheral neuron, dorsal root ganglion neuron, motor neuron, cortical neuron, hippocampal neuron, thalamic neuron, neuron of the cerebellum, excitatory neuron, inhibitory neuron, glutamatergic neuron, GABAergic neuron, and dopaminergic neuron. In some instances, the axon is of a neuron derived from a stem cell or neuronal progenitor cell. In other instances, the axon is of a neuron derived from a subject selected from the group consisting of a mouse, rat, dog, cat, pig, sheep, horse, non-human primate, and human. In yet other instances, the axon is of a neuron that is genetically modified to be resistant to an underlying pathology.

In yet another embodiment, the composition restores a lost or damaged axonal connection in the network.

In another aspect, the invention relates to a method of modulating the activity of a neurological network in a subject in need thereof, the method comprising: providing an elongated tubular construct having a first end and a second end, the construct comprising a tubular body having an outer surface and an inner surface defining a luminal core; positioning at least one neuron at the first end of the construct; culturing the neuron with the construct in vitro to promote extension of an axon of the neuron through at least a portion of the core, thereby forming a tissue-engineered composition; and administering the tissue-engineered composition into the subject.

In one embodiment, the method comprises positioning at least one neuron at the first end of the construct and positioning at least one neuron at the second end of the construct.

In another embodiment, the subject is selected from the group consisting of a mouse, rat, dog, cat, pig, sheep, horse, non-human primate, and human.

In yet another embodiment, the method restores an axonal connection in the central nervous system (CNS). Alternatively, the method restores an axonal connection in the peripheral nervous system (PNS). In one instance, the method replaces a lost, damaged, or degenerating neuron. In other instances, the method restores an axonal connection damaged as a result of a condition selected from the group consisting of traumatic brain injury, spinal cord injury, peripheral nerve injury, stroke, Alzheimer's disease, Parkinson's disease, Gulf War Illness, Huntington's disease, and ALS.

In yet another embodiment, the method modulates the activity of the network by creating additional synaptic inputs into the network or by releasing neurotransmitter to the network. In one instance, the method modulates dysfunctional activity of the network which underlies the pathology of a condition selected from the group consisting of seizure, epilepsy, depression, obesity, drug addiction, and Parkinson's disease.

In yet another embodiment, administering the composition comprises loading the composition into a needle and injecting the composition into a tissue of the subject. Alternatively, administering the composition comprises directly penetrating a tissue of the subject with the composition.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1D, depicts the results of experiments demonstrating the imaging of bi-directional micro-tissue engineered neural networks (Micro-TENNs). Confocal reconstructions of a bi-directional dorsal root ganglia (DRG) neuron Micro-TENN stained via immunocytochemistry to denote neuronal somata/dendrites (MAP-2), axons (β-tubulin III), and cell nuclei (Hoechst) at 31 DIV (FIG. 1A). Neuronal somata were restricted to dense ganglia on both ends of the Micro-TENN (FIG. 1B and FIG. 1D), while the interior of the Micro-TENN was comprised exclusively of long neurites (FIG. 1C). Scale bar (FIG. 1A)=500 μm. Scale bar (FIG. 1B-FIG. 1D)=100 μm.

FIG. 2, comprising FIG. 2A through FIG. 2C, depicts the results of experiments demonstrating neurite penetration into DRG neuron Micro-TENNs. Confocal reconstructions of Micro-TENNs at 7, 21, and 42 DIV. Constructs were stained via immunocytochemistry to denote neuronal somata/dendries (MAP-2), axons (β-tubulin III), and cell nuclei (Hoechst). At 7 DIV, neurites extended 3 mm into the Micro-TENN (FIG. 2A). At 21 DIV, neurites penetrated over 6 mm (FIG. 2B), and by 42 days, bi-directional axons had crossed to form a construct measuring approximately 2 cm (FIG. 2C). Scale bar (FIG. 2A)=200 μm. Scale bar (FIG. 2B)=300 μm. Scale bar (FIG. 2C)=800 μm.

FIG. 3, comprising FIG. 3A through FIG. 3F, depicts the results of experiments investigating cortical neuron Micro-TENN cytoarchitecture. Confocal reconstructions of a Micro-TENN plated with primary cerebral cortical neurons at 14 DIV. This Micro-TENN was stained via immunocytochemistry to denote neuronal somata/dendrites (MAP-2), axons (β-tubulin III), and cell nuclei (Hoechst). Seeded cortical neurons formed numerous clusters at the Micro-TENN ends, and extended neurites into the Micro-TENN interior (FIG. 3A). Discrete labeling for neuronal somata/dendrites, axons, and cell nuclei is shown in (FIG. 3B), (FIG. 3C), and (FIG. 3D), as well as their overlay in (FIG. 3E). Scale bar (FIG. 3A-FIG. 3E)=200 μm. FIG. 3F is a phase-contract image depicting a cortical neuron Micro-TENN with a discrete population neuron and long uni-directional axonal tracts.

FIG. 4, comprising FIG. 4A through FIG. 4G, depicts the results of experiments examining Micro-TENN implant survival. Confocal reconstructions of surviving implanted neurons at multiple time points. Cortical neurons were transduced in vitro with GFP (FIG. 4B-FIG. 4F). As indicated by the square (bottom left) in (FIG. 4A), surviving neurons from a transduced cortical Micro-TENN were found in the center of the Micro-TENN 7 days post-implant (FIG. 4B). As indicated by the square (upper right) in (FIG. 4A), surviving neuronal clusters from a transduced cortical Micro-TENN were found at the end of the Micro-TENN, near the cortical surface, 28 days post-implant (FIG. 4C-FIG. 4F). A 20 μm section was labeled via immunohistochemistry to denote neuronal somata/dendrites (MAP-2), axons (NF200), and cell nuclei (Hoechst). Labeling for neuronal somata/dendrites and axons confirmed that the phenotype of these surviving clusters was indeed neuronal (FIG. 4D-FIG. 4F). Confocal reconstructions of a DRG neuron Micro-TENN at 3 days following stercotaxic micro-injection to connect cortical to thalamic structures (FIG. 4G) (CGRP; projection from 20 μm thick section). Scale bar (FIG. 4B)=100 μm. Scale bar (FIG. 4C)=20 μm. Scale bar (FIG. 4D-FIG. 4F)=100 μm.

FIG. 5, comprising FIG. 5A through FIG. 5G, depicts the results of experiments investigating implanted cortical neuron Micro-TENN architecture and integration. Confocal reconstructions of a transduced cortical Micro-TENN 28 days post-implant. Neurons were transduced in vitro with GFP. As indicated by the rectangle (parallel to the Micro-TENN) in (FIG. 4A), deeper along the length of the Micro-TENN we found longitudinal projections at the Micro-TENN cortex interface (FIG. 5A-FIG. 5D). A 20 μm section was labeled via immunohistochemistry to denote neuronal synapses (synapsin), axons (NF200), and cell nuclei (Hoechst). These projections were comprised of implanted neurons (FIG. 5A) and aligned axons (FIG. 5B). Labeling with synapsin revealed the presence of synapsin-positive puncta along these radial projections, which suggests the possibility of synaptic inputs on the implanted neurites (FIG. 5C). As indicated by the rectangle (perpendicular to the Micro-TENN) in (FIG. 4A), it was also found that implanted neurons extended neurites into the host cortex (FIG. 5E-FIG. 5G). A 20 μm section was labeled via immunohistochemistry to denote neuronal synapses (synapsin). Higher magnification confocal reconstructions from a demonstrative region in (FIG. 5E) show putative dendritic spines along in-growing neurites, and synapsin-positive puncta in immediate proximity to these dendritic spines (FIG. 5F-FIG. 5G). Scale bar (FIG. 5A-FIG. 5E)=40 μm. Scale bar (FIG. 5F-FIG. 5G)=20 μm. * indicates host cortex.

FIG. 6 is an image depicting the results of experiments demonstrating that surviving neurons from a GFP+ cortical neuron Micro-TENN were found deeper along the length of the construct 28 days post-implant. Aligned neurites were observed along the cortical-thalamic axis (20 μm section).

FIG. 7 is a set of images depicting the results of experiments demonstrating that implanted neurons and axonal tracts appear uninhibited by host reactive astrocytes. A 20 μm section was labeled via immunohistochemistry to denote astrocytes (GFAP) and axons (NF200).

FIG. 8 is a set of images depicting the results of experiments demonstrating that implanted GFP+ Micro-TENN cortical neurons extended processes deep into the host cortex. In some cases, neurites penetrated up to several hundred microns (20 μm section).

FIG. 9, comprising FIG. 9A and FIG. 9B, depict exemplary schematics of the construct of the invention. FIG. 9A depicts a schematic of an exemplary bi-directional construct of the invention comprising an elongated tubular body with neurons at either end and axons extending bi-directionally through the luminal core of the construct. FIG. 9B depicts a schematic of an exemplary construct comprising a hydrogel tubular body and luminal core comprising a bioactive matrix.

FIG. 10 depicts a schematic of exemplary micro-tissue engineered neural networks (TENNs) of the present invention. Unidirectional (left) or bidirectional (right) Micro-TENNS may be utilized based on the characteristics of the axonal tract to be reconstructed. Prominent axonal pathways in the brain are shown, with examples of a potential replaced cortical-thalamic tract and the nigralstriatal pathway. The Micro-TENN design promoted neuronal survival at one or both ends while facilitating axon extension through conduit interior. In particular, these micro-scale tubes were generated using an agarose exterior (with varying concentrations and physical properties to provide structural support) and extracellular matrix interior (providing bioactive ligands: collagen, fibrin, and/or laminin) to encourage neuron survival, localization and longitudinal neurite extension while enabling minimally invasive injection for simultaneous neuronal and long-distance axonal tract replacement.

FIG. 11, comprising FIG. 11A and FIG. 11B, depicts schematics for exemplary constructs to provide “needle-less” delivery of the Micro-TENN into neural tissue. FIG. 11A depicts a strategy wherein the entire outer shell region comprises carboxymethylcellulose (CMC). FIG. 11B depicts a strategy wherein the agarose shell is coated with a CMC coating.

FIG. 12 is an image depicting Micro-TENNs lined up on micro-needles in preparation for the CMC-coating process.

FIG. 13, comprising FIG. 13A through FIG. 13F, is a series of images depicting the needle-less insertion of a tubular construct into surrogate “brain” material. An agarose construct coated with low-viscosity CMC and the surrogate brain material (gelatin), serves as a model of needle-less insertion into the brain. (FIG. 13A) Before initial insertion, the construct is straight, dry, and relatively rigid. (FIG. 13B) Upon contact with the gelatin, the construct remains hard and penetrates the surface of the gelatin that represents the brain. (FIG. 13C) After the completion of insertion, the construct is implanted in the gelatin for approximately 5 seconds. (FIG. 13D) The coated construct is removed from the brain showing that the construct is straight and the CMC is adhered to the construct. (FIG. 13E) For a second insertion attempt, the coated construct buckles since the CMC absorbed water from the gelatin during the first insertion, and therefore the construct has softened. Inset shows magnification of construct buckling. (FIG. 13F) After the failed insertion attempt, the coated construct is largely still intact, but slightly bent from buckling.

FIG. 14, comprising FIG. 14A through FIG. 14D, depicts the results of experiments examining the effects of agarose concentration (comprising the outer tubular structure) on cortical neuronal survival within Micro-TENNs. Phase contrast images of cortical neuronal clusters with dense neuritic extensions within the agarose microconduits (FIG. 14A and FIG. 14B). Confocal reconstruction showing an example of cortical neurons stained to denote live cells and neuritis (via calcium AM staining) (FIG. 14C). Graphical representation of the effects of microconduit agarose concentration on cortical neuronal survival within the Micro-TENNs (FIG. 14D). Neuronal survival was enhanced at 4% agarose compared to the other concentrations investigated.

FIG. 15, comprising FIG. 15A and FIG. 15B, depicts the detailed Micro-TENN concept. FIG. 15A depicts tissue-engineering construct (Micro-TENN) with discreet populations of cells at either end, connected via long-distance axonal connections. Micro-TENNs can be used to repair long-distance axonal pathways, depicted in tractography recreation of pathways in brain (FIG. 15A, right). Micro-TENN construct emulates axonal pathways with neuronal clusters at ends to allow for reintegration with native neurons (FIG. 15A, right, inset). FIG. 15B depicts needle versus needle-less insertion of Micro-TENNs into brain. Needle method (FIG. 15B, left) uses needle and plunger system to pierce brain surface then eject Micro-TENN into brain parenchyma. Needle-less method (FIG. 15B, right) uses CMC-coated Micro-TENN where needle is brought to surface of brain, and coated Micro-TENN penetrates brain surface and ultimately rests in brain parenchyma.

FIG. 16, comprising FIG. 16A through FIG. 16F, depicts ooverview diagram of Micro-TENN fabrication process. FIG. 16A depicts the Micro-TENN fabrication process starting with an empty capillary tube with an acupuncture needle through the center. FIG. 16B depicts that one end of the capillary tube is placed in warm agarose. Capillary action draws up the agarose. FIG. 16C depicts that after the agarose has cooled, one end of the construct is put in the matrix material that is desired to fill the core of the microconduit. FIG. 16D depicts the ends of the microconduit can be cut at a 45 degree angle (w.r.t. to plate bottom) in order to create a small trough for cells to be seeded. Seeded microconduits can be anchored in media via addition agarose struts or other methods. FIG. 16E depicts that after several days in vitro, neurons will show robust features and axonal outgrowth. FIG. 16F depicts that when the Micro-TENN has matured to the level desired, Micro-TENNs can be coated with the CMC-coating.

FIG. 17, comprising FIG. 17A through FIG. 17G, depicts Micro-TENN cytoarchitecture over time. FIG. 17A depicts representative cortical neuronal Micro-TENNs (750 μm OD, 350 μm ID; 3% agarose shell; 1 mg/ml collagen IV+1 mg/ml laminin core; 56,000 cells/ml) at 4 DIV. FIG. 17B depicts representative cortical neuronal Micro-TENNs (750 μm OD, 350 μm ID; 3% agarose shell; 1 mg/ml collagen IV+1 mg/ml laminin core; 72,000 cells/ml) at 4 DIV. FIG. 17C depicts representative cortical neuronal Micro-TENNs (750 μm OD, 350 μm ID; 3% agarose shell; 1 mg/ml collagen IV+1 mg/ml laminin core; 36,000 cells/ml) at 5 DIV. The primary cortical neurons were resuspended in Neurobasal media before plating. FIG. 17D depicts representative cortical neuronal Micro-TENNs (750 μm OD, 350 μm ID; 3% agarose shell; 1 mg/ml collagen IV+1 mg/ml laminin core; 36,000 cells/ml) at 5 DIV. The primary cortical neurons were resuspended in extracellular matrix solution (1 mg/ml collagen IV+1 mg/ml laminin) before plating. FIG. 17E depicts representative cortical neuronal Micro-TENNs (750 μm OD, 350 μm ID; 3% agarose shell; 1 mg/ml collagen IV+1 mg/ml laminin core; 36,000 cells/ml) at 7 DIV. FIG. 17F depicts representative cortical neuronal Micro-TENNs (750 μm OD, 350 μm ID; 3% agarose shell; 1 mg/ml collagen IV+1 mg/ml laminin core; 36,000 cells/ml) at 8 DIV. FIG. 17G depicts representative cortical neuronal Micro-TENNs (750 μm OD, 350 μm ID; 3% agarose shell; 1 mg/ml collagen IV+1 mg/ml laminin core; 36,000 cells/ml) at 18. Scale bars=100 μm.

FIG. 18, comprising FIG. 18A through FIG. 18F, depicts long-term cellular phenotype and Micro-TENN architecture. FIG. 18A depicts DAPI stained representative cortical neuronal Micro-TENN (750 μm OD, 350 μm ID; 3% agarose shell; 1 mg/ml collagen IV, 1 mg/ml laminin core; 36,000 cells/ml) fixed at 18 DIV. Scale bars=100 μm. FIG. 18B depicts MAP2 stained representative cortical neuronal Micro-TENN (750 μm OD, 350 μm ID; 3% agarose shell; 1 mg/ml collagen IV, 1 mg/ml laminin core; 36,000 cells/ml) fixed at 18 DIV. Scale bars=100 μm. FIG. 18C depicts TUJ1 stained representative cortical neuronal Micro-TENN (750 μm OD, 350 μm ID; 3% agarose shell; 1 mg/ml collagen IV, 1 mg/ml laminin core; 36,000 cells/ml) fixed at 18 DIV. Scale bars=100 μm. FIG. 18D depicts 3D-projection of Micro-TENN from channel opening (left of image) to mid-tube (right of image) at 10×. Scale bars=100 μm. FIG. 18E depicts representative nucleus of MAP2-positive cells projecting axonal tracts in multiple directions at 40×. Scale bars=25 μm. FIG. 18F depicts laterally projecting TUJ1-positive axonal tracts at 40×. Scale bars=25 μm.

FIG. 19, comprising FIG. 19A through FIG. 19C, depicts mechanical testing of buckling force and brain insertion force. FIG. 19A depicts representative trace from CMC-coated Micro-TENN driven into solid aluminum block. FIG. 19B depicts representative trace from a stainless steel wire (diameter similar to CMC-coated Micro-TENN) inserted into 0.6% agarose (brain phantom). FIG. 19C is a graph depicting forces from CMC-coated Micro-TENN buckling trials and steel wire into agarose insertion trials. Error bars are standard deviation.

FIG. 20, comprising FIG. 20A though FIG. 20J, depicts snapshots of insertion of steel wire and CMC-coated Micro-TENN into brain phantom. FIGS. 20A-D depict the different stages of insertion when steel wire (similar size to CMC-coated Micro-TENN) was implanted into 0.6% agarose (brain phantom). FIGS. 20E-J depict snapshots of insertion of CMC-coated Micro-TENN into brain phantom.

### DETAILED DESCRIPTION OF THE INVENTION

The present invention relates to compositions and methods for modulating the activity of neurological networks. In one embodiment, the invention provides for neuronal replacement and reestablishing damaged or lost axonal connections. In one embodiment, the invention provides for the neuromodulation of existing networks. For example, the present invention is partly based upon the manufacture of tissue-engineered constructs that support neuronal survival and axonal extension and the finding that implantation of the constructs promoted proper synaptic integration of the constructs and restoration of long-distance axonal connections.

In one embodiment, the invention includes a composition comprising an elongated tubular construct, comprising a tubular body defining a luminal core. In one embodiment, the tubular body is a hydrogel. In one embodiment, the luminal core comprises one or more extracellular matrix proteins to support the growth and extension of axonal projections. In certain embodiments, the outer surface of the tubular body is coated with a layer to provide mechanical strength and rigidity to the construct, allowing the construct to be delivered directly into a tissue region in need. In one embodiment, the coating softens upon implantation to match with the mechanical properties of the surrounding environment of the tissue.

In one embodiment, the invention provides a method of modulating the activity of a neurological network. In one embodiment the method comprises reestablishing damaged or lost axonal connections. In one embodiment, the method comprises adding additional synaptic inputs into an existing network. The method comprises administering the construct of the invention to a tissue of the central nervous system (CNS) or peripheral nervous system (PNS) in need thereof. In certain instances, the method is useful for treating any disease, disorder, or condition characterized by axonal loss or damage, including, but not limited to, brain injury, spinal cord injury, peripheral nerve injury, Gulf War Veterans Illness, stroke and neurodegenerative diseases, including Parkinson's disease, ALS, Huntington's disease, and Alzheimer's disease. In one embodiment, the method is useful for treating any disease, disorder, or condition characterized by pathological or dysfunction network activity, including, but not limited to seizure, epilepsy, depression, obesity, drug addiction, and Parkinson's disease. In one embodiment, the method comprises culturing the construct of the invention with a neuron, neural progenitor cell, or stem cell ex vivo under appropriate conditions to promote axonal extension within the luminal core of the substrate. In certain embodiments, the method comprises administering the resulting tissue-engineered composition to a tissue region in need thereof.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, +10%, +5%, +1%, or +0.1% from the specified value, as such variations are appropriate to perform the disclosed methods.

The terms “precursor cell,” “progenitor cell,” and “stem cell” are used interchangeably in the art and as used herein refer either to a pluripotent or lineage-uncommitted progenitor cell, which is potentially capable of an unlimited number of mitotic divisions to either renew itself or to produce progeny cells which will differentiate into the desired cell type. In contrast to pluripotent stem cells, lineage-committed progenitor cells are generally considered to be incapable of giving rise to numerous cell types that phenotypically differ from each other. Instead, progenitor cells give rise to one or possibly two lineage-committed cell types.

The term “dedifferentiation,” as used herein, refers to the return of a cell to a less specialized state. After dedifferentiation, such a cell will have the capacity to differentiate into more or different cell types than was possible prior to re-programming. The process of reverse differentiation (i.e., de-differentiation) is likely more complicated than differentiation and requires “re-programming” the cell to become more primitive.

As used herein, “scaffold” refers to a structure, comprising a biocompatible material, that provides a surface suitable for adherence and proliferation of cells. A scaffold may further provide mechanical stability and support. A scaffold may be in a particular shape or form so as to influence or delimit a three-dimensional shape or form assumed by a population of proliferating cells. Such shapes or forms include, but are not limited to, films (e.g., a form with two-dimensions substantially greater than the third dimension), ribbons, cords, sheets, flat discs, cylinders, spheres, 3-dimensional amorphous shapes, etc.

As used here, “biocompatible” refers to any material, which, when implanted in a mammal, does not provoke an adverse response in the mammal. A biocompatible material, when introduced into an individual, is not toxic or injurious to that individual, nor does it induce immunological rejection of the material in the mammal.

As used herein, “autologous” refers to a biological material derived from the same individual to whom the material will later be re-introduced.

As used herein, “allogeneic” refers to a biological material derived from a genetically different individual of the same species as the individual to whom the material will be introduced.

As used herein, a “graft” refers to a cell, tissue or organ that is implanted into an individual, typically to replace, correct or otherwise overcome a defect. A graft may further comprise a scaffold. The tissue or organ may consist of cells that originate from the same individual; this graft is referred to herein by the following interchangeable terms: “autograft,” “autologous transplant,” “autologous implant” and “autologous graft.” A graft comprising cells from a genetically different individual of the same species is referred to herein by the following interchangeable terms: “allograft,” “allogeneic transplant,” “allogeneic implant” and “allogeneic graft.” A graft from an individual to their identical twin is referred to herein as an “isograft,” a “syngeneic transplant,” a “syngeneic implant” or a “syngeneic graft.” A “xenograft,” “xenogeneic transplant” or “xenogeneic implant” refers to a graft from one individual to another of a different species.

As used herein, to “alleviate” a disease, defect, disorder or condition means reducing the frequency and/or severity of one or more signs and/or symptoms of the disease, defect, disorder or condition experienced by subject.

As used herein, to “treat” means reducing the frequency and/or severity with which signs and/or symptoms of a disease, defect, disorder, or adverse condition, and the like, are experienced by a patient.

As used herein, a “therapeutically effective amount” is the amount of a composition of the invention sufficient to provide a beneficial effect to the individual to whom the composition is administered.

As used herein, a “therapeutic treatment” is a treatment administered to a subject for the purpose of treating, curing, preventing, or diminishing or eliminating one or more symptoms of a disease, defect, disorder or condition. As used herein, a “therapeutically effective amount” is the amount of a composition of the invention sufficient to provide a beneficial effect to the individual to whom the composition is administered. As used herein, “treating a nerve lesion” is used interchangeably with “treating a nerve injury” and means repairing the injured nerve region or reducing the frequency and/or the severity of a sign and/or symptom of the nerve lesion. As used herein, the term “nerve injury” and “nerve lesion” are used interchangeably and refer to any damage or disruption of the neuronal axons.

As used herein, the term “growth medium” is meant to refer to a culture medium that promotes growth of cells. A growth medium will generally contain animal serum. In some instances, the growth medium may not contain animal serum.

“Differentiation medium” is used herein to refer to a cell growth medium comprising an additive or a lack of an additive such that a stem cell, fetal neural progenitor cell or other such progenitor cell, that is not fully differentiated, develops into a cell with some or all of the characteristics of a differentiated cell when incubated in the medium.

As used herein, the term “growth factor” refers to a protein, peptide, mitogen, or other molecule having a growth, proliferative, differentiative, or trophic effect on a cell. Growth factors include, but are not limited to, fibroblast growth factor (FGF), basic fibroblast growth factor (bFGF), acidic fibroblast growth factor (aFGF), epidermal growth factor (EGF), insulin-like growth factor-I (IGF-T), insulin-like growth factor-II (IGF-II), platelet-derived growth factor (PDGF), vascular endothelial cell growth factor (VEGF), activin-A, bone morphogenic proteins (BMPs), insulin, growth hormone, erythropoietin, thrombopoietin, interleukin 3 (IL-3), interleukin 6 (IL-6), interleukin 7 (IL-7), macrophage colony stimulating factor, c-kit ligand/stem cell factor, osteoprotegerin ligand, insulin, nerve growth factor, ciliary neurotrophic factor, cytokines, chemokines, morphogens, neutralizing antibodies, other proteins, and small molecules. Preferably, the FGF is selected from the group selected from FGF2, FGF7, FGF10, and any combination thereof.

As used herein, “in vitro” and “ex vivo” are used interchangeably to refer to conditions existing outside of the body of a living organism. Thus, in vitro culturing and ex vivo culturing both refer to culturing outside of the body of a living organism.

An “isolated cell” refers to a cell which has been separated from other components and/or cells which naturally accompany the isolated cell in a tissue or mammal.

As used herein, a “substantially purified” group of cells is a group of two or more cells of one cell type that is essentially free of other cell types. Thus, a substantially purified group of cells refers to a group of two or more cells which has been purified from other cell types with which they are normally associated in their naturally-occurring state.

“Expandability” is used herein to refer to the capacity of a cell to proliferate, for example, to expand in number or, in the case of a population of cells, to undergo population doublings.

As used herein, “tissue engineering” refers to the process of generating tissues ex vivo for use in tissue replacement or reconstruction. Tissue engineering is an example of “regenerative medicine,” which encompasses approaches to the repair or replacement of tissues and organs by incorporation of cells, gene or other biological building blocks, along with bioengineered materials and technologies.

As used herein “endogenous” refers to any material from or produced inside an organism, cell or system.

“Exogenous” refers to any material introduced into or produced outside an organism, cell, or system.

The term “tissue,” as used herein includes, but is not limited to, bone, nervous tissue, fibrous connective tissue including tendons and ligaments, cartilage, dura, pericardia, muscle, lung, heart valves, veins and arteries and other vasculature, dermis, adipose tissue, or glandular tissue.

As used herein, the terms “subject,” “patient” and “individual” are used interchangeably. As used herein, a subject is preferably a mammal such as a non-primate (e.g., cows, pigs, horses, cats, dogs, rats, etc.) or a primate (e.g., monkey and human), most preferably a human.

As used herein, “synapse” refers to a junction between a neuron and another cell, across which chemical communication flows. As used herein, “synapsed” refers to a neuron that has formed one or more synapses with one or more cells, such as another neuron or a muscle cell. As used herein, “synaptically integrate” refers to the formation of at least one synapse between a neuron and at least one other cell. The other cell may be a nerve cell, a muscle cell or another neuron target. For instance, two neurons are synaptically integrated if at least one synapse exists between the two cells.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides a composition and method for modulation of the activity of neurological networks. In one embodiment, the invention provides neuronal replacement and reestablishing long-distance axonal connections. For example, in certain embodiments the present invention is used to restore or replace long-distance axonal connections that are lost as a result of a disease, disorder, or condition including, but not limited to traumatic brain injury, spinal cord injury, stroke, peripheral nerve injury, Gulf War Veterans Illness, and neurodegenerative disorders, including for example Parkinson's disease, ALS, Huntington's disease, and Alzheimer's disease.

In one embodiment, the invention comprises replacement of neurons that are lost as a result of a disease, disorder, or condition including, but not limited to traumatic brain injury, spinal cord injury, stroke, peripheral nerve injury, Gulf War Veterans Illness, and neurodegenerative disorders, including for example Parkinson's disease, ALS, Huntington's disease, and Alzheimer's disease.

In one embodiment, the invention provides for modulation of existing neural networks. For example, in certain embodiments, the invention allows for modulating the existing neural activity in one or more brain regions of a normal or diseased brain. That is, the invention allows for delivery of inhibitory neurons to dampen network activity, or delivery of excitatory neurons to stimulate network activity. This may be used, for example in treating conditions associated with overactive network activity (e.g., seizure, epilepsy, etc.) or for conditions treatable by increasing local neural network activity (e.g., depression, obesity, addiction, etc.). Thus, the present invention includes a method of modulating underlying neural activity in a disease or disorder associated with pathological or dysfunctional neural network activity by administering the construct of the invention into the region in need. The administered neuron of the construct can integrate with the existing network to modulate activity in order to treat the disease or disorder.

In one embodiment, the invention comprises tissue-engineered compositions constructed ex vivo and implantation of said compositions into the central or peripheral nervous system to replace lost neurons or restore lost connections.

In one embodiment, the invention includes a composition for promoting the repair, restoration, or replacement of long-distance axonal connections. In certain embodiments, the composition replaces lost, damaged, or degenerating, neurons. In one embodiment, the composition comprises an elongated tubular construct comprising a body surrounding a luminal core. In certain embodiments, the construct is pre-formed in vitro prior to delivery into a subject. The construct is referred to herein as “Micro-TENN.” In certain embodiments, the composition comprises at least one axon tract within the core, where the tract runs longitudinally for at least part of the length of the construct. In one embodiment the body comprises a hydrogel. In one embodiment, the core comprises a structural protein, or fragment thereof, including, for example, extracellular matrix proteins including, collagen, laminin, hyaluronic acid, fibrin, fibronectin, or the like. For example, in certain embodiments, the core region comprises a matrix through which one or more axon tracts extend. The present invention is partly based upon the development of micro-tissue engineering techniques which generate discrete neuronal populations with long integrated axonal tracts encased in tubular hydrogels. For example, as described elsewhere herein, up to 1 month post-implant, the constructs maintained their neuronal architecture with axonal fascicles extending parallel to cortical-thalamic projections. In addition, cortical neurons from the micro-constructs extended neurites into host cortex, with immunohistochemical evidence of synaptic integration.

In one embodiment, the body, core, or both comprise one or more compounds, including for example, proteins, nucleic acids, small molecules, hormones, growth factors, and the like, which enhance axonal growth, promote survival, reduce host inflammation, or promote integration of the composition into host tissue.

In one embodiment, the body comprises a coating layer which allows for needle-less delivery of the construct.

In one embodiment, the invention provides a method for reestablishing a lost axonal connection comprising administering a Micro-TENN construct of the invention to a region in which one or more axonal connections has been lost. The Micro-TENN replaces lost or damaged neurons and physically recreates axonal tracts. In certain instances, the neurons of the Micro-TENN synapse locally, and thus the pre-formed axonal tracts serves as a new relay.

In certain instances, the method is useful for treating any disease, disorder, or condition characterized by axonal loss or damage, including, for example, brain injury, spinal cord injury, peripheral nerve injury, stroke and neurodegenerative diseases, including Parkinson's disease, ALS, Huntington's disease, and Alzheimer's disease. In one embodiment, the method comprises ex vivo manufacture of a Micro-TENN followed by implantation of the Micro-TENN in a region in need. In certain embodiments, the dimensions of the construct allow for minimally invasive implantation into the brain. In certain embodiments, ex vivo manufacture of a Micro-TENN comprises placing at least one neuron or stem cell at one end of the tubular construct, as described herein, and promoting the growth and extension of an axon through the luminal core of the tubular construct. In one embodiment, the method comprises placing at least one neuron or stem cell at a first end of the elongated tubular construct and at least one neuron or stem cell at a second end of the elongated tubular construct. In certain embodiments, promoting the growth and extension of axons from both neurons forms bi-directional Micro-TENNs.

### Composition

The present invention includes a composition for reestablishing damaged or lost axonal connections. For example, in certain embodiments the composition is used to restore or replace lost or damaged axons in the central or peripheral nervous systems. In one embodiment, the composition, when implanted into a region in need thereof, forms proper connections with existing axons, neurons, or synapses.

In one embodiment, the composition comprises an elongated tubular member construct, having a first and second end. In one embodiment, the construct comprises a tubular body having an outer surface and inner surface defining a luminal core. The length of the construct, defined by the distance between the first end and second end, may be any suitable length, and in certain instances is dependent upon the end use of the construct. For example, in one embodiment, the length of the tubular construct is about 0.1 mm to about 10 cm.

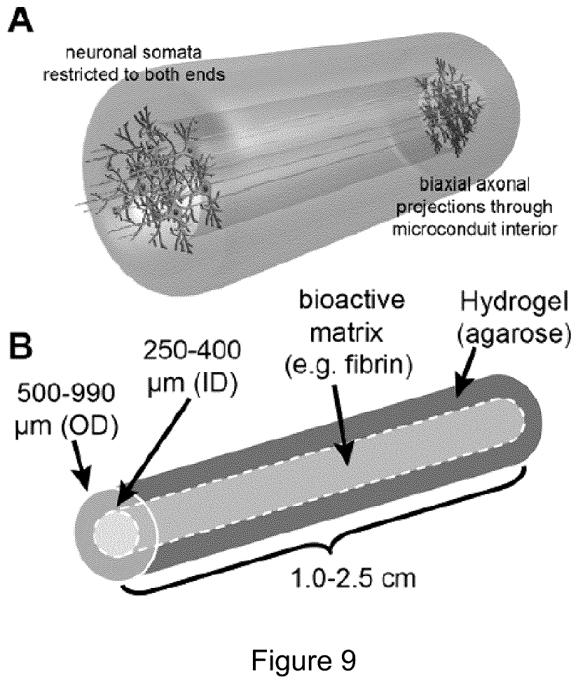

The construct has an outer diameter and an inner diameter, where the outer diameter is the diameter of the construct, while the inner diameter is the diameter of the luminal corc. The diameters of the construct may be any suitable diameter to promote growth and survival of an axon while allowing for safe and efficient delivery into a region in need. For example, in one embodiment the outer diameter is chosen to provide non-invasive or minimally invasive delivery. For example, in certain embodiments, the miniature dimensions of the construct allow for minimally invasive delivery. Further, in certain embodiments the outer diameter is dependent upon the desired degradation time of the implanted construct. In one embodiment, the outer diameter is about 10 μm to about 10 mm. In one embodiment, the outer diameter is about 500 μm to about 1 mm. The inner diameter of the construct may be dependent, for example, upon the desired amount or density of axons extending through the construct. In one embodiment, the inner diameter of the construct is about 1 μm to about 1 mm. In one embodiment, the inner diameter of the construct is about 125 μm to about 500 μm. A schematic depicting an exemplary construct of the invention is shown in FIG. 9.

In certain embodiments, the construct is biocompatible. For example, in one embodiment, the construct, when implanted, does not generate an adverse immunogenic or inflammatory response in the subject. In certain embodiments, the tubular body of the construct degrades over time thereby leaving the encapsulated axon tracts within the subject.

In one embodiment, the construct comprises a hydrogel. For example, in certain embodiments, the body of the construct comprises a hydrogel. Hydrogels can generally absorb a great deal of fluid and, at equilibrium, typically are composed more than about 60% fluid and less than about 40% polymer. In a preferred embodiment, the water content of hydrogel is about 80-99.9%. Hydrogels are particularly useful due to the inherent biocompatibility of the cross-linked polymeric network (Hill-West, et al., 1994, Proc. Natl. Acad. Sci. USA 91:5967-5971). Hydrogel biocompatibility can be attributed to hydrophilicity and ability to imbibe large amounts of biological fluids (Preparation and Characterization of Cross-linked Hydrophilic Networks in Absorbent Polymer Technology, Brannon-Peppas and Harland, Eds. 1990, Elsevier: Amsterdam, pp 45-66; Preparation Methods and Structure of Hydrogels in Hydrogels in Medicine and Pharmacy, Peppas, Ed. 1986, CRC Press: Boca Raton, Fla., pp 1-27). The hydrogels can be prepared by crosslinking hydrophilic biopolymers or synthetic polymers. Examples of the hydrogels formed from physical or chemical crosslinking of hydrophilic biopolymers, include, but are not limited to, hyaluronans, chitosans, alginates, collagen, dextran, pectin, carrageenan, polylysine, gelatin or agarose. (Hennink and van Nostrum, 2002, Adv. Drug Del. Rev. 54, 13-36 and Hoffman, 2002, Adv. Drug Del. Rev. 43, 3-12). These materials consist of high-molecular weight backbone chains made of linear or branched polysaccharides or polypeptides. Examples of hydrogels based on chemical or physical crosslinking synthetic polymers include, but are not limited to, (meth)acrylate-oligolactide-PEO-oligolactide-(meth)acrylate, poly(ethylene glycol) (PEO), poly(propylene glycol) (PPO), PEO-PPO-PEO copolymers (Pluronics), poly(phosphazene), poly(methacrylates), poly(N-vinylpyrrolidone), PL (G) A-PEO-PL (G) A copolymers, poly(ethylene imine), etc. (Hoffman, 2002, Adv. Drug Del. Rev, 43, 3-12). In some embodiments, the hydrogel comprises poly(ethylene glycol) diacrylate (PEGDA).

In one embodiment, the hydrogel comprises at least one biopolymer. In other embodiments, the hydrogel scaffold further comprises at least two biopolymers. In yet other embodiments, the hydrogel scaffold further comprises at least one biopolymer and at least one synthetic polymer.

In one embodiment, the hydrogel comprises agarose. The concentration of agarose may, in certain instances, be dependent upon the type of neuron ultimately being cultured, the mechanical properties, desired, or the like. For example, it is described herein, that increasing concentrations of agarose enhances neuronal survival and neurite outgrowth. In one embodiment, the concentration of agarose is about 0.1% to about 10%. In one embodiment, the concentration of agarose is about 0.5% to about 5%. In one embodiment, the concentration of agarose is about 4%.

Hydrogels closely resemble the natural living extracellular matrix (Ratner and Hoffman. Synthetic Hydrogels for Biomedical Applications in Hydrogels for Medical and Related Applications, Andrade, Ed. 1976, American Chemical Society: Washington, D.C., pp 1-36). Hydrogels can also be made degradable in vivo by incorporating PLA, PLGA or PGA polymers. Moreover, hydrogels can be modified with fibronectin, laminin, vitronectin, or, for example, RGD for surface modification, which can promote cell adhesion and proliferation (Heungsoo Shin, 2003, Biomaterials 24:4353-4364; Hwang et al., 2006 Tissue Eng. 12:2695-706). Indeed, altering molecular weights, block structures, degradable linkages, and cross-linking modes can influence strength, elasticity, and degradation properties of the instant hydrogels (Nguyen and West, 2002, Biomaterials 23 (22): 4307-14; Ifkovits and Burkick, 2007, Tissue Eng. 13 (10): 2369-85).

Molecules which can be incorporated into the hydrogel matrix, for example via covalent linkage, encapsulation, or the like, include, but are not limited to, vitamins and other nutritional supplements; glycoproteins (e.g., collagen); fibronectin; peptides and proteins; carbohydrates (both simple and/or complex); proteoglycans; antigens; oligonucleotides (sense and/or antisense DNA and/or RNA); antibodies (for example, to infectious agents, tumors, drugs or hormones); and gene therapy reagents. Hydrogels may be modified with functional groups for covalently attaching a variety of proteins (e.g., collagen) or compounds such as therapeutic agents. Therapeutic agents which can be incorporated to the matrix include, but are not limited to, analgesics, anesthetics, antifungals, antibiotics, anti-inflammatoires, anthelmintics, antidotes, antihistamines, antihypertensives, antimalarials, antimicrobials, antipsychotics, antipyretics, antiseptics, antiarthritics, antituberculotics, antivirals, chemotherapeutic agents, a colored or fluorescent imaging agent, corticoids (such as steroids), antidepressants, depressants, diagnostic aids, enzymes, hormones, hypnotics, minerals, nutritional supplements, parasympathomimetics, potassium supplements, radiation sensitizers, a radioisotope, sedatives, sulfonamides, stimulants, sympathomimetics, tranquilizers, vasoconstrictors, vasodilators, vitamins, xanthine derivatives, and the like. The therapeutic agent may also be other small organic molecules, naturally isolated entities or their analogs, organometallic agents, chelated metals or metal salts, peptide-based drugs, or peptidic or non-peptidic receptor targeting or binding agents. It is contemplated that in certain embodiments, linkage of the therapeutic agent to the matrix may be via a protease sensitive linker or other biodegradable linkage.

In certain embodiments, one or more multifunctional cross-linking agents may be utilized as reactive moieties that covalently link biopolymers or synthetic polymers. Such bifunctional cross-linking agents may include glutaraldehyde, epoxides (e.g., bis-oxiranes), oxidized dextran, p-azidobenzoyl hydrazide, N-[α.-malcimidoacetoxy]succinimide ester, p-azidophenyl glyoxal monohydrate, bis-[β-(4-azidosalicylamido)ethyl]disulfide, bis [sulfosuccinimidyl]suberate, dithiobis [succinimidyl proprionate, disuccinimidyl suberate, 1-ethyl-3-[3-dimethylaminopropyl]carbodiimide hydrochloride (EDC), N-hydroxysuccinimide (NHS) and other bifunctional cross-linking reagents known to those skilled in the art. It should be appreciated by those skilled in the art that the mechanical properties of the hydrogel are greatly influenced by the cross-linking time and the amount of cross-linking agents.

In another embodiment utilizing a cross-linking agent, polyacrylated materials, such as ethoxylated (20) trimethylpropane triacrylate, may be used as a non-specific photo-activated cross-linking agent. Components of an exemplary reaction mixture would include a thermoreversible hydrogel held at 39° C., polyacrylate monomers, such as ethoxylated (20) trimethylpropane triacrylate, a photo-initiator, such as cosin Y, catalytic agents, such as 1-vinyl-2-pyrrolidinone, and triethanolamine. Continuous exposure of this reactive mixture to long-wavelength light (>498 nm) would produce a cross-linked hydrogel network.

In one embodiment, the hydrogel comprises a UV-sensitive curing agent which initiates hydrogel polymerization. For example, in one embodiment, a hydrogel comprises the photoinitiator 4-(2-hydroxyethoxy)phenyl-(2-hydroxy-2-propyl) ketone. In one embodiment, polymerization is induced by 4-(2-hydroxyethoxy)phenyl-(2-hydroxy-2-propyl) ketone upon application of UV light. Other examples of UV sensitive curing agents include 2-hydroxy-2-methyl-1-phenylpropan-2-one, 4-(2-hydroxyethoxy)phenyl (2-hydroxy-2-phenyl-2-hydroxy-2-propyl)ketone, 2,2-dimethoxy-2-phenyl-acetophenone 1-[4-(2-Hydroxyethoxy)-phenyl]-2-hydroxy-2-methyl-1-propane-1-one, 1-hydroxycyclohexylphenyl ketone, trimethyl benzoyl diphenyl phosphine oxide and mixtures thereof.

The stabilized cross-linked hydrogel matrix of the present invention may be further stabilized and enhanced through the addition of one or more enhancing agents. By “enhancing agent” or “stabilizing agent” is intended any compound added to the hydrogel matrix, in addition to the high molecular weight components, that enhances the hydrogel matrix by providing further stability or functional advantages. Suitable enhancing agents, which are admixed with the high molecular weight components and dispersed within the hydrogel matrix, include many of the additives described earlier in connection with the thermoreversible matrix discussed above. The enhancing agent can include any compound, especially polar compounds, that, when incorporated into the cross-linked hydrogel matrix, enhance the hydrogel matrix by providing further stability or functional advantages.

Preferred enhancing agents for use with the stabilized cross-linked hydrogel matrix include polar amino acids, amino acid analogues, amino acid derivatives, intact collagen, and divalent cation chelators, such as ethylenediaminetetraacetic acid (EDTA) or salts thereof. Polar amino acids are intended to include tyrosine, cysteine, serine, threonine, asparagine, glutamine, aspartic acid, glutamic acid, arginine, lysine, and histidine. The preferred polar amino acids are L-cysteine, L-glutamic acid, L-lysine, and L-arginine. Suitable concentrations of each particular preferred enhancing agent are the same as noted above in connection with the thermoreversible hydrogel matrix. Polar amino acids, EDTA, and mixtures thereof, are preferred enhancing agents. The enhancing agents can be added to the matrix composition before or during the crosslinking of the high molecular weight components.

The enhancing agents are particularly important in the stabilized cross-linked bioactive hydrogel matrix because of the inherent properties they promote within the matrix. The hydrogel matrix exhibits an intrinsic bioactivity that will become more evident through the additional embodiments described hereinafter. It is believed the intrinsic bioactivity is a function of the unique stereochemistry of the cross-linked macromolecules in the presence of the enhancing and strengthening polar amino acids, as well as other enhancing agents.

In one embodiment, the luminal core of the construct comprises one or more extracellular matrix components to promote axonal survival and extension through the construct. For example, in certain embodiments, the inner surface of the body is coated with one or more extracellular matrix component. In one embodiment, the core comprises a dense matrix comprising one or more extracellular matrix component.

Exemplary extracellular matrix components include, but are not limited to, collagen, fibronectin, fibrin, hyaluronic acid, elastin, laminin, and the like. In certain instances, the type of extracellular matrix component within the core is dependent upon the type of neuron or axon being cultured with the construct. For example, in certain embodiments, a construct comprising collagen within the core supports the extension of axonal projections from DRG neurons. In certain embodiments, a construct comprising collagen, laminin, fibrin, or a combination thereof, within the core supports extension of axonal projections from cerebral cortical neurons.

In one embodiment, the density of the one or more extracellular matrix components within the core is optimized for the survival of the neuron and extension of the axon. The appropriate density of the one or more extracellular matrix components may vary based upon the identity of the component, combination of components, desired extension rate, or the like. In certain embodiments, the density of the one or more extracellular component is about 1 μg/mL to about 100 mg/mL.

In one embodiment, the core of the construct comprises one or more compounds, including, for example, small molecules, proteins, peptides, growth factors, hormones, nucleic acids, and the like, which enhance axonal growth and survival within the core. For example, in certain embodiments, nerve growth factor (NGF), brain-derived neurotrophic factor (BDNF), or a combination thereof may be coated or administered into the core, which in certain instances will improve neuronal health and axonal elongation through the construct.

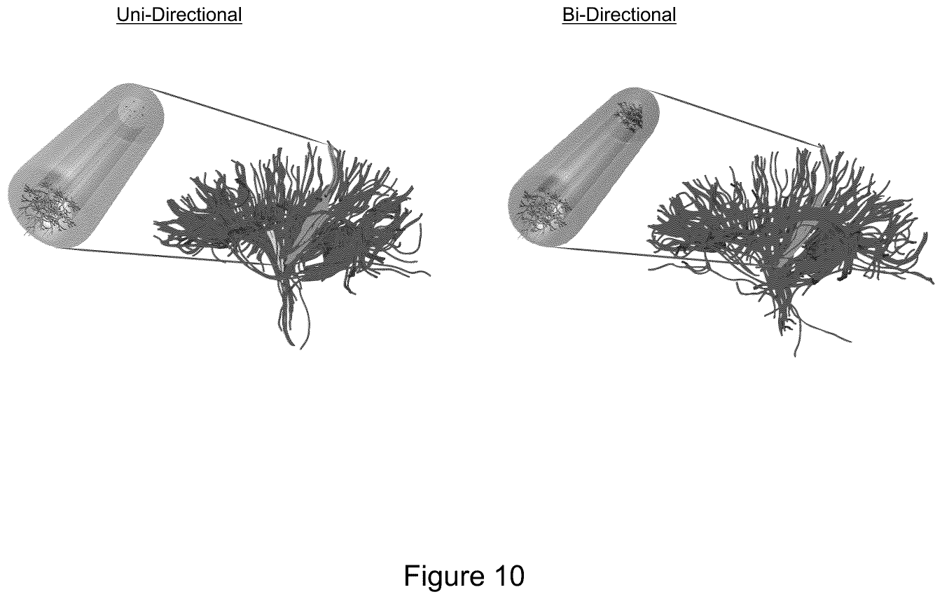

In one embodiment, the composition of the invention comprises a construct, as described herein, and a cell. For example, in certain embodiments, the composition comprises an elongated tubular construct having at least one axon extending through the core of the construct. The present invention is partly based upon the finding that positioning a neuron at the end of the construct and incubating the construct and neuron under appropriate conditions in vitro or ex vivo, promotes axonal growth and extension through the construct. In one embodiment, the composition comprises a construct having uni-directional axonal extension, as would be generated by culturing a neuron at one end of the construct (FIG. 10). In one embodiment, the composition comprises a construct having bi-directional axonal extension, as would be generated by culturing at least one neuron at the first end of the construct and at least one neuron at the second end of the construct (FIG. 9A, FIG. 10). In certain instances, bi-directional axonal extension through the construct allows for increased length of total axon extension and construct length.

The neurons useful for the compositions and methods provided herein include all neuronal subtypes, including but not limited to PNS motor or sensory, CNS, and stem cells (e.g., induced pluripotent stem cells, embryonic stem cells, and the like) differentiated into a neuronal phenotype. In one embodiment of the present invention, neurons are derived from any cell that is a neuronal cell (e.g., cortical neurons, dorsal root ganglion neurons or sympathetic ganglion neurons) or is capable of differentiating into a neuronal cell (e.g., stem cell). The neurons may be autologous, allogenic, or xenogenic with reference to the subject.

In certain embodiments, the neurons are peripheral or spinal cord neurons including dorsal root ganglion neuron or motor neuron. In certain embodiments, the neurons are from brain, including but not limited to, neurons from the cerebral cortex, thalamus, hippocampus, and cerebellum. The neurons may be excitatory or inhibitory neurons. The neurons may be glutamatergic, dopaminergic, GABAergic, or any other type of neuron as classified based upon its primary neurotransmitter.

Neurons useful in the invention may be derived from cell lines or other mammalian sources, such as donors or volunteers. In one embodiment, the neurons are human neurons. In one embodiment, the neurons are non-human mammalian neurons, including neurons obtained from a mouse, rat, dog, cat, pig, sheep, horse, or non-human primate. In one embodiment, the neurons are cortical neurons, hippocampal, neurons, dorsal root ganglion neurons or sympathetic ganglion neurons. In another embodiment, neurons are derived from immortalized cell lines that are induced to become neuron-like (e.g., NT2, PC12). In one embodiment, the neurons are neurons derived from a cadaver. In another embodiment, the neurons are neurons derived from patients who have undergone ganglionectomies or other clinical procedure. Furthermore, the neurons may be singular, integrated neurons or a plurality of integrated neurons (i.e., an integrated nerve bundle).

In one embodiment, the neuron is genetically modified. For example, in certain embodiments the neuron is modified to add or subtract genetic material to modify RNA or protein expression. In certain embodiments, the neuron is modified to render the neuron resistant to an underlying pathology, including but not limited to, amyloidosis or tauopathy in Alzheimer's disease or synucleinopathies in Parkinson's disease. The neuron and construct is designed to recapitulate the lost neuroanatomy characteristic of a given disease, while simultaneously being designed to be resistant to the pathology of the disease. For example, the neuron may be designed to allow for the clearance of pathological proteins, including but not limited to alpha-synuclein, amyloid-beta, and tau. In one embodiment, the neuron is designed to bolster axonal transport of proteins by combating transport interruptions, providing continuity of the cytoskeleton, or bolster expression or activity of motor proteins. In one embodiment, the neuron is designed to improve energetics by boosting ATP production or creatine synthesis. In another embodiment, the neuron is designed to boost pro-survival cues, including but not limited to extracellular matrix proteins and neurotropic factors. For example, neurons may be modified to express a neurotrophic factor. In some embodiments, expression of a neurotrophic factor is transient. In some embodiments, expression of a neurotrophic factor is stable. Methods of genetically modifying neurons, such as viral transduction/transfection and electroporation, are well known to the skilled artisan.

The number or density of the cells positioned at either end of the construct is dependent upon the type of neuron being used and the eventual use of the construct. For example, in certain embodiments, 1, 100, 1000, 10000, 1000000, 100000000, or more cells are positioned at an end of the construct.

In certain embodiments, the in vitro or ex vivo culture of the constructs with the neurons is done under suitable conditions to promote the growth of axons through the core of the construct. Those conditions include, without limitation, the appropriate temperature and/or pressure, electrical and/or mechanical activity, force, the appropriate amounts of O₂ and/or CO₂, an appropriate amount of humidity, and sterile or near-sterile conditions. For example, the cells may require a nutritional supplement (e.g., nutrients and/or a carbon source such as glucose), exogenous hormones or growth factors, and/or a particular pH. Exemplary cell culture media that can support the growth and survival of the neuron includes, but is not limited to, Neurobasal, Neurobasal A, Dulbecco's Modified Eagle Medium (DMEM), and Minimum Essential Medium (MEM). In certain embodiments, the culture medium is supplemented with B-27. In certain embodiments, the culture medium may contain fetal bovine serum or serum from another species at a concentration of at least 1% to about 30%, or about 5% to about 15%, or about 10%. In one embodiment, the culture medium comprises Neurobasal supplemented with about 2% B-27 and about 500 μM L-glutamine.

In certain embodiments, the in vitro or ex vivo culture of the construct is conducted for an appropriate time period to allow for extension of an axon through the length of the construct. Appropriate time period of culture will depend upon the length of the construct, type of neuron used, culture conditions, and the like. Determination of when to remove the constructs from culture may be aided by use of standard experimental procedures including, but not limited to, electrophysiological recordings, immunohistochemistry, fluorescent imaging, calcium imaging, transduction using channel rhodopsins, and the like.

In one embodiment, the outer surface of the body of the construct is coated. For example, in one embodiment, the coating provides sufficient mechanical strength and rigidity to allow the construct to be directly inserted or injected into a region of interest without the need for a needle. For example, the coated construct can directly penetrate the brain without using a metal needle, thereby providing for less-invasive “needle-less” delivery. In certain embodiments, the coating is altered upon insertion into the region of interest, which softens the construct when placed. That is, the coating provides hydration-dependent mechanical properties to the construct, where it is rigid when not hydrated or mildly hydrated, and soft when hydrated. For example, the material softens to match the mechanical properties of the brain. This prevents unnecessary neuroinflammation and gliosis due to the chronic mechanical mismatch between the construct and the surrounding brain tissue, which is seen with implanted rigid neuroelectrodes. In one embodiment, the thickness of the coating is about 0.1 μm to about 100 μm. In one embodiment, the thickness of the coating is about 15 μm.

In one embodiment, the coating comprises carboxymethylcellulose (CMC). CMC is a cellulose derivative with carboxymethyl groups bound to hydroxyl groups. The functional properties depend on degree of substitution of cellulose structure and degree of polymerization. CMC possesses unique properties in that it is stiff in a dehydrated state and gel-like hydrated state, with a short transition period between states at micro-dimensions. Also, CMC is nontoxic to humans and animals, inexpensive, and widely available.

In certain embodiments, the coating is applied to the construct by first applying dry CMC to the outer surface of the construct and then applying a liquid to the dry CMC. This application procedure forms a uniform CMC coating on the outer surface of the substrate.

The coating allows for the “needless” delivery of the construct into a desired tissue or region. For example, in one embodiment, the delivery comprises loading the coated construct into a needle, and using a plunger to eject the construct out of the tip in order to engage the tissue surface. The rigidity of the coated construct allows for the construct to penetrate the tissue surface, thereby delivering the construct into the desired tissue.

### Treatment Methods

The present invention provides a method of treating a disorder, disease, or condition in a subject in need thereof by modulating or repairing a neuronal network on the subject. For example, in one embodiment the method comprises reestablishing one or more lost axonal connections in the subject. In one embodiment, the method comprises modulating the existing neuronal network of the subject in order to modulate its functional activity.

In certain embodiments the method of the invention restores, replaces, or reproduces lost axonal connections. For example, the present invention comprises administering a tissue-engineered construct comprising living axonal tracts to a region or tissue in need thereof. In certain embodiments, the present invention is used to restore axonal connections in regions or tissues in the CNS or PNS where axonal connections have been lost due to injury or disease.

It is contemplated that the methods of the present invention will be applicable to reestablish lost axonal connections in the CNS. For example, the method of the invention restores or replaces axons that have been damaged or lost due to injury or disease. As described herein, administration of the construct of the invention restores brain circuitry. For example, the construct becomes integrated into the network of the region by forming proper synaptic connections with the existing neurons. By synaptically integrating into the neural network, brain circuitry is restored and functional activity and behavior is improved.

In certain embodiments, the method of the invention modulates the function of existing neuronal circuitry in the brain. For example, in one embodiment, the method comprises administering a Micro-TENN, as described elsewhere herein, in order to add one or more neuronal inputs into an existing network, thereby influencing the functional activity of the network.

In one embodiment, the method influences the strength of connections. For example, in one embodiment the method comprises administering to a brain region in need, one or more Micro-TENN designed to provide inhibitory inputs to pathways that are exerting too much (i.e. too strong) influence causing detrimental functional effects in certain disease states. In one embodiment, inhibitory Micro-TENNs act by forming inhibitory (e.g., GABAergic) synapses to the existing network. In another embodiment, the inhibitory Micro-TENNs act by bulk release of inhibitory neurotransmitter (e.g., GABA) by the axonal terminal. In certain embodiments, the administered Micro-TENNs may exert tonic (i.e. continuous, self-pacing) activity or respond to inputs from the existing host circuitry of the subject.

In one embodiment, the method comprises administering to a brain region in need, one or more Micro-TENN designed to provide excitatory inputs to pathways that are exerting too little (i.e. too weak) influence causing detrimental functional effects in certain disease states. In one embodiment, inhibitory Micro-TENNs act by forming excitatory (e.g., glutamatergic) synapses to the existing network. In another embodiment, the excitatory Micro-TENNs act by bulk release of excitatory neurotransmitter (e.g., glutamate) by the axonal terminal. In certain embodiments, the administered Micro-TENNs may exert tonic (i.e. continuous, self-pacing) activity or respond to inputs from the existing host circuitry of the subject.

Neuromodulation of existing networks, as conducted by way of the present method, would be useful for treating a variety of conditions, including, but not limited to seizures, epilepsy, Parkinson's disease, depression, obesity, drug addiction, and the like. The present method allows for the permanent “dial up” or “dial down” (i.e. increase or decrease the gain) of specific components of a given circuit involved in a particular behavior.

For example, in one embodiment, the method comprises treating intractable seizures. Currently, treatment of seizures which do not respond to pharmacological agents comprises the ablation or surgical removal of the seizure foci. The present invention allows for use of inhibitory Micro-TENNs to suppress the activity of the foci or circuitry involved in the seizure. This inhibition could be tonic or programmed to respond to the earliest signs of epileptiform activity, for example by release of inhibitory neurotransmitter. In one embodiment, neuromodulation of the network may be used for treating conditions in which deep brain stimulation (DBS) is currently being used or proposed. In certain instances, neuromodulation by way of Micro-TENNs is advantageous over DBS, because Micro-TENNs are smaller, living, permanent, and capable of responding to the host conditions. In contrast, DBS uses 1.0-1.5 mm diameter electrodes, which may trigger an immune response. Further, DBS uses additional bulky hardware and battery, and the system must initially be tuned and often requires re-tuning and recharging.

Diseases or conditions that may be treated using the methods of the present invention include those affecting the CNS, the PNS, or a combination thereof. The CNS includes the brain, spinal cord, optic, olfactory and auditory systems. The PNS includes neurons and nervous tissue that reside or extend outside of the CNS.

In certain embodiments, the present method is used to treat subjects with spinal cord injury, traumatic brain injury, stroke, as well as other nerve lesions, such as those derived from a neurodegenerative disease or peripheral neuropathy. Exemplary neurodegenerative diseases treatable by way of the present method include, but are not limited to, Alzheimer's disease, Parkinson's disease, Huntington's disease, Creutzfeldt-Jakob disease, Gulf War Illness, and ALS. The present method is also useful for treating neurodegeneration as a result of aging and drug use.

The term “peripheral neuropathy” refers to damage to the peripheral nerve system that is caused by an inherited disease, physical trauma, tumor, toxin, infection, autoimmune response, nutritional deficiency, vascular disorder, or metabolic disorder. Diseases or infectious organisms that may cause peripheral neuropathy include, but are not limited to, acute inflammatory demyelinating neuropathy, Guillain-Barré syndrome, chronic inflammatory demyelinating polyneuropathy (CIDP), Charcot-Marie-Tooth disease, amyloid polyneuropathy, multifocal motor neuropathy, Lyme disease, diptheria, leprosy, human immunodeficiency virus (HIV), herpes varicella-zoster, Epstein-Barr, cytomegalovirus, and herpes simplex viruses. The compositions and methods of the invention may also be used to restore the neural connections of a severed limb, organ or tissue with the central nervous system. In another embodiment, the lesion is an optic nerve lesion. In another embodiment, the lesion is any peripheral nerve lesion, including upper and lower limb, genitourinary nerve and craniofacial nerve damage.

In one embodiment, the method comprises treating a nerve injury, including, for example, an injury to a peripheral nerve of the subject. In one embodiment, the injury to the peripheral nerve is caused by a peripheral neuropathy. In one embodiment, the injury to the peripheral nerve is caused by trauma. In one embodiment, the injury to the peripheral nerve is caused by cancer. In one embodiment, the injury to the peripheral nerve is caused by a surgery. In one embodiment, the injury to the peripheral nerve is a congenital anomaly. In another embodiment, the nerve injury comprises an injury to the spinal cord of a subject. In another embodiment, the nerve injury is caused by a disease or condition that may include, for example, amyotrophic lateral sclerosis, carpal tunnel syndrome, or any other disease or condition relating to a loss of motor or sensory nerve function. In one embodiment, the nerve injury is caused by an amputation. In one embodiment, the nerve injury is caused by complete or partial removal of an organ, tumor, or tissue. In one embodiment, the nerve injury comprises the complete transection of the nerve. In one embodiment, the nerve injury comprises the compression or crushing of a nerve or nerve segment. For example, in one embodiment, the nerve injury comprises an acute crush injury or a repetitive crush injury, or an acute compression injury or a repetitive compression injury. In one embodiment, the crush or compression injury creates or contributes to a distal environment that is non-permissive or resistant to axonal regeneration. In one embodiment, the nerve injury occurs during or as a result of a medical procedure, including a surgical procedure. For example, nerve injury may occur due to crushing, compression, bruising, inflammation, or transection of a nerve during surgery. For example, nerve injury may occur as a result of contacting the nerve with the scalpel; bruising, inflammation, stretching, crushing, or compression of the nerve due to contact with surgical equipment and/or patient positioning during surgery (for example, prolonged Trendelenburg positioning or brachial injury in laparascopic injury due to positioning of patient arms). In one embodiment, nerve injury can occur as a result of radiation therapy (e.g., brachial plexopathy) or chemotherapy. In one embodiment, nerve injury can occur as a result of metabolic/endocrine complications including diabetes; inflammatory and autoimmune diseases; vitamin deficiencies including vitamins B6 and B12; infectious diseases including Lyme disease, herpes viruses, HIN and hepatitis C; and toxic causes, such as alcoholism. In one embodiment, nerve injury can occur as a result of accidental exposure to organic metals and heavy metals (for example, lead, arsenic and mercury). In one embodiment, nerve injury can occur as a result of drugs (for example, heart and blood pressure medications such as, for example, amiodarone, hydralazine and perphexiline; drugs used to treat infections such as, for example, chloroquine, isoniazid, metronidazole, nitrofurantoin, thalidomide; drugs used to treat autoimmune disease such as, for example, etanercept, infliximab and leflunomide; drugs used to treat skin conditions such as, for example, dapasone; anticonvulsants such as, for example, phehytoin; anti-alcohol drugs such as, for example disfulfiram; drugs to fight HIV such as, for example, didanosine, stavudine and zalcitabine; and colchicine).

In one embodiment, the method comprises treating a spinal cord injury or traumatic brain injury. Spinal cord injury, includes partial or complete transection of the spinal cord, crush injury of the spinal cord, or compression of the spinal cord. Traumatic brain injury includes penetrating or non-penetrating brain injuries. Traumatic brain injury includes injuries suffered as a result of trauma, repetitive head trauma, blast injury, and the like. In certain instances, traumatic brain injury results in immediate or progressive loss of axonal connections. The method further comprises restoring axonal connections in the CNS, in a region where axons have been damaged or lost as result of tumor resection, for example in treating a brain tumor, acoustic neuroma or optic nerve meningioma. In certain embodiments, the method is used to restore axonal connections in any brain region, including, but not limited to, the cortex, hippocampus, thalamus, hypothalamus, midbrain, cerebellum, and the like. In certain embodiments, the method restores axonal connections between brain regions.

In one embodiment, the method of the invention comprises administering the construct, described elsewhere herein, to replace or restore a damaged or lost axon having a length of about 0.1, about 0.5, about 1.0, about 1.5, about 2.0, about 2.5, about 3.0, about 3.5, about 4.0, about 4.5, about 5.0, about 6.0, about 7.0, about 8.0, about 9.0, about 10.0, or more centimeters (cm) in length.

In one embodiment, the method comprises administering the construct by inserting (e.g., injecting, etc.) the construct to a region in need. For example, in certain embodiments, the miniature dimensions of the construct allows for minimally invasive insertion (e.g., injection, etc.) into the brain, spinal cord, or peripheral nerve. In one embodiment, administration comprises positioning the construct within a needle, advancing the needle to a region of need, and injecting the construct. In another embodiment, the construct comprises a coating which provides the construct with the mechanical strength to penetrate the tissue on its own, thereby providing “needle-less” delivery of the construct.

Determination of the functional integration of the constructs with the host tissue may be aided using standard experimental procedures including, but not limited to, electrophysiological recordings, immunohistochemistry, fluorescent imaging, calcium imaging, transduction using channel rhodopsins, and the like.

In one embodiment, the method comprises administering a plurality of constructs, as described herein, to the region in need. For example, in certain embodiments, the implanted plurality of constructs may form a network of connections within the region to restore neural circuitry and functional activity.

In certain embodiments, the method comprises providing one or more compounds to the region in need thereof to aid in the growth, survival, and function of the implanted construct. Such compounds include, for example, neurotrophic factors, growth factors, hormones, small molecules, and the like. In one embodiment, the method comprises administering a therapeutic agent, including, for example, an anesthetic, analgesic, steroid, anti-inflammatory agent, or the like, to the region in need. In certain embodiments, compounds or therapeutic agents are administered by intra-nerve, intravenous, intraperitoneal, intracerebral, intramuscular, intraocular, intraarterial, intralesional, or topical routes. In one embodiment, the inflammatory response may be modulated upon delivery in order to potentially improve construct survival and integration with the brain.

The methods provided may be used with any subject having or suspected of having a disease, disorder, or condition associated with a loss of axonal connections. In one embodiment, the subject is a mammal. In a further embodiment, the subject is a veterinary animal, such as, but not limited to, non-human primates, horses, cattle, sheep, dogs, cats, pigs, and goats. In another embodiment, the subject is a human.

In one embodiment, the invention includes a method of stimulating an existing network in the subject, recording from an existing network in the subject, or a combination thereof. For example, the Micro-TENNs of the present invention may be used as “living biological electrodes” that allows for long-term stimulation and recording not possible using traditional electrodes. In one embodiment, the administered Micro-TENNs have one end penetrating the cerebral cortex, or deeper structures, and with the other end at or near the cortical surface. In certain instances, this would obviate the need for penetrating stimulating electrodes or penetrating recording electrodes, which have a relatively short useful life due to bio-fouling, glial scar formation, and reduced neuronal density in the vicinity of the electrodes.

In certain embodiments, this allows for biological modulation of the region or circuitry of interest as controlled by an external computer or other hardware. For example, brain surface electrodes or transcranial electric or magnetic stimulation can be used to stimulate the Micro-TENN neurons, which would then relay the stimulation to modulate the activity of some deeper brain structure.

In one embodiment, the administered Micro-TENNs allow for recording of deep neural activity from the surface. In one embodiment, the neurons of the Micro-TENNs integrate with host neurons, and relay host activity to the surface where recording electrodes detect the activity. In combination, stimulation and recording using Micro-TENNs form the backbone of a strategy of biologically-based brain-computer interface, with the advantage that the component that penetrates the brain is biological and hence has the possibility of permanently integrating and not ultimately being rejected as a foreign body.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Micro-Tissue-Engineered Neural Networks with Preformed Cytoarchitecture Survive and Integrate with the Cerebral Cortex

As a strategy to simultaneously replace lost neurons and restore long-distance axonal connections, micro-Tissue Engineered Neural Networks (TENNs) were created. These constructs comprise discrete neuronal populations with long integrated axonal tracts encased in miniature tubular hydrogels (roughly three times the diameter of a human hair and extending up to several centimeters in length). The exterior hydrogel shell contains an interior extracellular matrix core to support neurite extension. The inner core was made with either collagen or fibrin. The densities of these hydrogels were optimized to control the movement of the cell bodies and create distinct somatic and axonal regions within the Micro-TENNs. Dissociated embryonic rat neurons were precisely delivered to one or both ends of the micro-constructs, which were cultured for 7-140 days in vitro (DIV) based on desired length of axon growth. Neurons remained in discrete populations on the Micro-TENN ends, and demonstrated robust longitudinal axonal extensions along the internal column. Notably, this general geometry recapitulates the basic systems-level functional architecture of the brain: discrete neuronal populations spanned by long axonal tracts.

The Micro-TENNs were also designed with miniature dimensions to permit minimally invasive implantation into the brain. In order to assess neuronal survival and axonal integration, pre-formed Micro-TENNs were stereotaxically injected, which resulted in the demonstration of dense axonal outgrowth to connect deep thalamic structures with the cerebral cortex in rats. Whereas prior studies have implanted fetal grafts, single-cell suspensions, or cells in three dimensional (3-D) matrices (Denham et al., 2012, Front Cell Neurosci 6:11; Fawcett et al., 1995, Exp Brain Res, 106 (2): 275-282; Mine et al., 2013, Neurobiol Dis 52:191-203; Ren et al., 2013, Int J Clin Exp Pathol 6 (2): 230-241; Sinclair et al., 1999, Eur J Neurosci 11 (12): 4341-4348; Tate et al., 2009 J Tissue Eng Regen Med 3 (3): 208-217; Tate et al., 2002, Cell Transplant 11 (3): 283-295; Yoo et al., 2011, Exp Neurobiol 20 (2): 110-115), the present method was considerably different in that it involved generating the final cytoarchitecture of the Micro-TENN in vitro and implanting it en masse. While constructs were developed using both dorsal root ganglia and primary cortical neurons in vitro, the implant study described herein primarily utilized cortical Micro-TENNs.

Overall, the micro-tissue engineered constructs described herein represent the first approach to facilitate nervous system repair by simultaneously replacing lost neurons and re-creating long-distance axonal connections in the brain. The studies presented herein examined how to improve the Micro-TENN design, characterized the rate of axonal penetration into the constructs, and demonstrated Micro-TENN survival in vivo out to one month.

The materials and methods employed in these experiments are now described.

### 3D Micro-TENN Fabrication

All supplies were from Invitrogen (Carlsbad, CA) or BD Biosciences (San Jose, CA) unless otherwise noted. Micro-TENNs were comprised of an agarose-extracellular matrix (ECM) hydrogel molded into a cylinder through which axons could grow. The outer hydrogel structure consisted of 1-4% agarose (Sigma-Aldrich, St. Louis, MO) in DPBS. The cylinder, with an outer diameter that ranged from 500 μm to 990 μm, was generated by drawing the heated agarose solution into a microliter glass capillary tube (Drummond Scientific, Broomall, PA) via capillary action. An acupuncture needle (diameter: 250 μm) (Seirin, Weymouth, MA) was inserted in the center of the liquid agarose-filled capillary tube in order to produce an inner column. Once the capillary tubes had cooled to room temperature, they were dipped into a liquid collagen or fibrin solution (rat tail type I collagen, 3.0 mg/ml or salmon fibrin, 1.0 mg/mL fibrinogen with 0.5 units/mL thrombin (Reagent Proteins, San Diego, CA)). A negative-pressure gradient was created by slowly retracting the needle, which drew the solution into a central column (i.e., clear of agarose). The Micro-TENNs, now comprised of an agarose tubular shell with a collagen or fibrin core, were then incubated at 37° C. for 30 minutes. The cured Micro-TENNs were gently pushed out of the capillary tubes and placed in DPBS where they were cut to 4-35 mm in length and sterilized under UV light (15 minutes). Following sterilization, collagen Micro-TENNs were plated with dorsal root ganglia neurons and fibrin Micro-TENNs were plated with cerebral cortical neurons.

### Neuronal Cell Culture

Dorsal root ganglia (DRG) were isolated from embryonic day 16 Sprague-Dawley rats (Charles River, Wilmington, MA). Carbon dioxide was used to euthanize time-pregnant rats, following which the uterus was extracted through Caesarian section. Each fetus was removed from the amniotic sac and put in cold Leibovitz-15 medium. The spinal cords were dissected out and individual DRG were plucked using forceps. To dissociate the explants, DRGs were placed in pre-warmed trypsin (0.25%)+EDTA (1 mM) and were incubated at 37° C. for 1 hour. Following the addition of Neurobasal medium+5% FBS, the tissue was triturated and then centrifuged at 1000 rpm for 3 minutes. The supernatant was aspirated, and the cells were resuspended at 5×10⁶ cells/mL in Neurobasal medium+2% B-27+500 μM L-glutamine+1% FBS (Atlanta Biologicals)+2 mg/mL glucose (Sigma)+10 ng/ml 2.5S nerve growth factor+10 mM FdU (Sigma) and 10 mM uridine (Sigma). Using a micropipette, approximately 5-10 μL of cell solution was precisely delivered to one or both ends of the Micro-TENNs (n=144). The cultures were placed in a humidified tissue culture incubator (37° C. and 5% CO₂) for 75 minutes to allow cells to attach, after which, media was added to the culture vessel. Fresh, pre-warmed media was used to change the culture media every 2-3 days in vitro (DIV).

Primary cerebral cortical neurons were isolated from embryonic day 18 Sprague-Dawley rats (Charles River, Wilmington, MA). Carbon dioxide was used to euthanize time-pregnant rats, after which the uterus was extracted through Caesarian section. Each fetus was removed from the amniotic sac and put in cold HBSS medium. The brains were removed and cerebral cortices were isolated. To dissociate the tissue, the cortices were placed in pre-warmed trypsin (0.25%)+EDTA (1 mM) for 12 minutes at 37° C. The trypsin-EDTA was then removed and the tissue was triturated in HBSS containing DNase I (0.15 mg/mL). The cells were centrifuged at 1000 rpm for 3 minutes and resuspended at 30×10⁶ cells/mL in neuronal plating medium (Neurobasal medium+2% B27+0.4 mM L-glutamine). Using a micropipette, approximately 5-10 μL of cell solution was precisely delivered to one or both ends of the Micro-TENNs (n=72). The cultures were placed in a humidified tissue culture incubator (37° C. and 5% CO₂) for 50 minutes to allow cells to attach, after which, media was added to the culture vessel. Fresh, pre-warmed media was used to change the culture media every 2-3 days in vitro (DIV).

### Transduction

In some instances, Micro-TENNs were transduced with an AAV viral vector (AAV2/1.hSynapsin.EGFP.WPRE.bGH) to produce GFP expression in the neurons. At 5 DIV, the Micro-TENNs were incubated overnight in media containing the viral vector (3.2×10¹⁰ Genome copies/mL). A media change was performed the following day.

### Immunocytochemistry

Micro-TENNs were fixed in 4% formaldehyde for 35 min, rinsed in PBS, and permeabilized using 0.3% Triton X100 plus 4% horse serum for 60 minutes. Primary antibodies were added (in PBS+4% serum) at 4° C. for 12 hours. The primary antibodies were the following neuronal markers: (1) MAP-2 (AB5622, 1:100, Millipore, Billerica, MA; AB5392, 1:100, Abcam, Cambridge, MA), a microtubule-associated protein expressed primarily in neuronal somata and dendrites and (2) β-tubulin III (T8578, 1:500, Sigma-Aldrich, Saint Louis, MO), a microtubule element expressed primarily in neurons. After rinsing, Alexa 488 donkey anti-mouse, Alexa donkey anti-rabbit 649, Alexa donkey anti-chicken 649, Alexa donkey anti-rabbit 568, Alexa donkey anti-chicken 594 IgG secondary antibodies (1:500 in 30 nM Hoechst in PBS+4% serum) were added at 18-24° C. for 2 hours.

### Implantation of Micro-TENNs

Adult male Sprague-Dawley rats (n=12) were maintained under isoflurane anesthesia. Once anesthetized, rats were mounted in a stereotactic frame (Kopf) and the skull was visualized. A craniotomy was performed alongside the temporal ledge, lateral to the midsagittal suture, and interior to both lambda and bregma sutures to access the sensory cortex. Immediately prior to delivery, a 4 mm Micro-TENN was drawn into a 16 gauge needle in warmed 1×DPBS. The needle was centered at 4.0 mm posterior to bregma, and adjacent to the temporal ledge. The needle was then lowered 6.0 mm at an 11 degree angle over 2 minutes to target the SI barrel cortex (Paxinos and Watson, 2006, The Rat Brain in Stereotaxic Coordinates, 6th Edition, Academic Press). A glass plunger was engaged within the needle to secure the Micro-TENN within the cortex while the needle was slowly retracted over 2 minutes. Once the Micro-TENN delivery was complete, the craniotomy sight was covered with sterilized Teflon tape that was secured to the skull with bone wax (Ethicon). The scalp was then sutured with 4-0 prolene sutures and the animals were recovered. Post-surgical analgesia included meloxicam (2.0 mg/kg) and bupivicane (2.0 mg/kg) at time of surgical recovery as well as 24 hours post Micro-TENN delivery. Animals receiving DRG Micro-TENNs were allowed to live for 3 days (n=2) after Micro-TENN delivery, while animals receiving cortical Micro-TENNs were allowed to live for either 7 days (n=4) or 28 days (n=4). At the time of sacrifice, animals were anesthetized with Euthasol and underwent transcardial perfusion with 0.1% heparinized saline followed by 4% paraformaldehyde. Animals were decapitated and post-fixed in 4% paraformaldehyde overnight to avoid handling artifacts.

### Immunohistochemistry

After post-fixing, brains were blocked off from bregma-6 to bregma+3. The blocked samples were placed in a 30% sucrose solution for a minimum of two days or until they saturated with sucrose. Tissue samples were flash frozen in isopentane at −42° C. and frozen to a chuck with OCT. Sections (20 μm) were cut and plated on Fisherbrand precleaned superfrost plus microscope slides and stored at −20° C. Slides were rinsed with PBS, dipped in 70% ethanol, and blocked in 2% normal horse serum for 35 minutes. Primary antibodies were applied (in Optimax solution) overnight at 4° C. The primary antibodies were specific to the following neuronal markers: (1) SMI31 (SMI-31R, 1:1500, Covance, Princeton, NJ), a 200 kDa neurofilament protein, (2) MAP2 (ab5392, 1:50, abcam, Cambridge, MA), a microtubule-associated protein primarily in neuronal somata and dendrites, (3) synapsin I, (A6442, 1:50, Invitrogen, Grand Island, NY), a phosphoprotein associated with the cytoplasmic surface of synaptic vesicles, and (4) GFAP, (ab53554, 1:50, abcam, Cambridge, MA), an intermediate filament-associated protein expressed in glial cells. After rinsing, Alexa 568 goat anti-rabbit, Alexa 568 donkey anti-mouse, Alexa donkey anti-goat 594, Alexa donkey anti-rabbit 649, Alexa donkey anti-mouse 649, and/or Alexa donkey anti-chicken 649 IgG secondary antibodies (1:500 in Optimax solution) were added at 18-24° C. for 1 hour. Slides were coverslipped using ProLong Gold w/DAPI (Life Technologies, Grand Island, NY).

### Microscopy, Data Acquisition, and Analyses

For intermediate in vitro analysis, Micro-TENNs were imaged using phase-contrast or fluorescence on a Nikon Eclipse Ti-S microscope with digital image acquisition using a QiClick camera interfaced with Nikon Elements BR 4.10.01. In order to determine the rate of neurite penetration, the longest observable neurite in each Micro-TENN was measured at 7, 14, 21, 28, 35, and 42 DIV (n=5 Micro-TENNs, repeated measurements). For terminal in vitro analyses, Micro-TENNs were fluorescently imaged using a laser scanning confocal microscope (LSM 710; Zeiss, Oberkochen, Germany) on a Zeiss Axio Observer microscope (Zeiss, Oberkochen, Germany). For each in vitro Micro-TENN analyzed via confocal microscopy, multiple z-stacks were digitally captured and analyzed. All confocal reconstructions were from full thickness z-stacks (≥250 μm when imaging cells within the inner diameter; ≥500-1000 μm when imaging clusters of neuronal somata). General Micro-TENN cytoarchitecture was qualitatively assessed at 7, 14, 21, 32, and 42 DIV for DRG and cortical neurons (n=6).

For analysis of Micro-TENNs post-transplant into the brain, Micro-TENNs were fluorescently imaged using a laser scanning confocal microscope (LSM 710; Zeiss, Oberkochen, Germany) on an Zeiss Axio Observer microscope (Zeiss, Oberkochen, Germany). For each 20 μm Micro-TENN section analyzed via confocal microscopy, multiple z-stacks were digitally captured and analyzed.

The results of the experiments are now described.

### Development of Bi-Directional Micro-TENNs

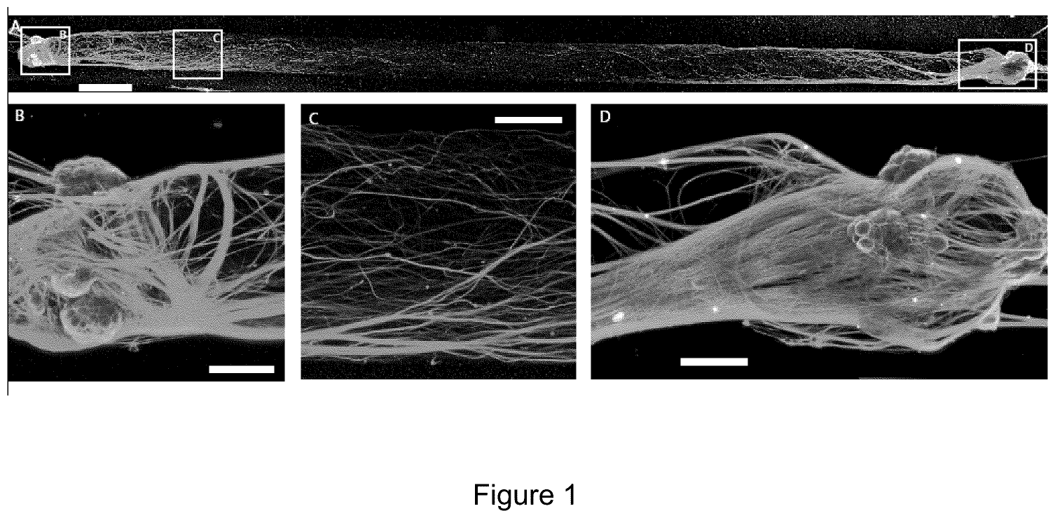

Following initial studies developing uni-directional Micro-TENNs constructs (Cullen et al., 2012, Tissue Engineering Part A, 18 (21-22): 2280-2289), bi-directional constructs were developed. Here, neuronal populations were plated on both ends of the Micro-TENN and allowed to extend neurites into the Micro-TENN interior (FIG. 10). As with the uni-directional constructs, the neuronal somata of the bi-directional constructs remained in dense ganglia, restricted to the Micro-TENN ends in most cases. These ganglia projected long neurites into the interior which, given sufficient time in vitro, overlapped and grew along each other (FIG. 1). The neurites primarily extended along the border between the extracellular matrix core and the agarose walls.

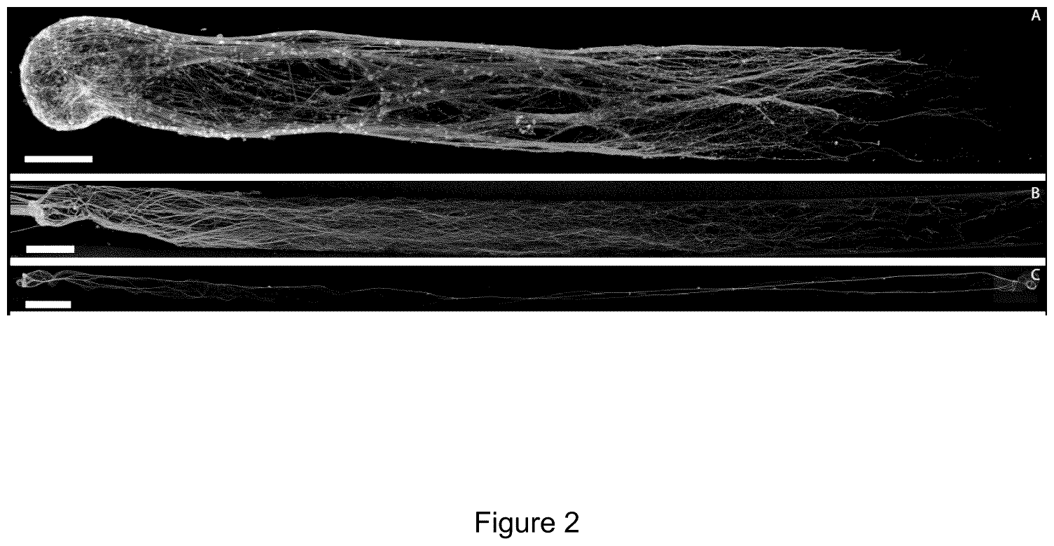

The length of neurite penetration in the Micro-TENNs was measured at weekly time points, and the average neurite length at each time point was calculated. Representative Micro-TENNs were fixed at 7, 21, and 42 DIV, and labeled via immunocytochemistry with MAP-2, beta tubulin III, and Hoechst to denote neuronal somata/dendrites, axons, and cell nuclei, respectively (FIG. 2). The Micro-TENNs exhibited 3 mm of ingrowth at 7 DIV, and by 42 DIV, the axonal projections had completely crossed to form a construct measuring approximately 2 cm. The majority of the ingrowth occurred by 4 weeks in vitro, after which the growth rate dropped to nearly 0 mm/day. Overall, the average rate of axonal extension through the interior was 0.16 mm/day over 6 weeks.

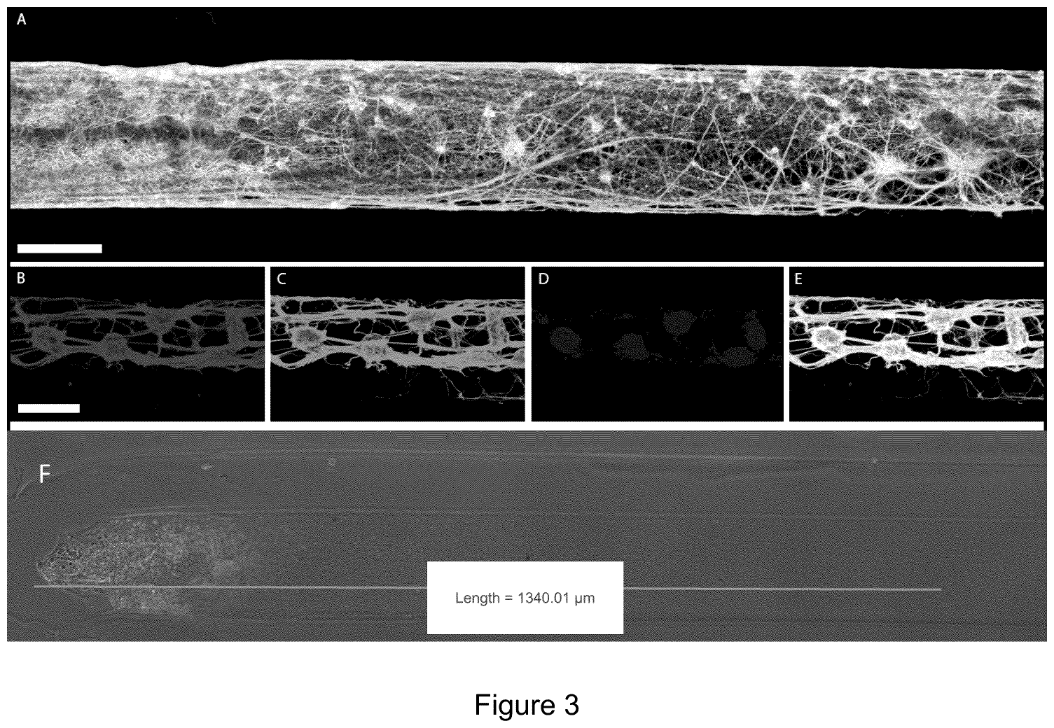

Micro-TENNs that utilized primary neurons from the cerebral cortex were also developed. It was found that collagen Micro-TENNs did not adequately support cortical neuronal survival and axon extension (estimated to be <10% viable). Following experimentation with different ECMs for the inner core, it was found that the cortical neurons exhibited the most robust health and outgrowth in a salmon-derived fibrin matrix or a blend of collagen+laminin (each estimated to yield >75% viability). Besides the replacement of collagen with fibrin or collagen+laminin, the fabrication methods for these cortical Micro-TENNs remained the same as for the DRG Micro-TENNs. Rather than assembling in a singular, dense ganglion at the end of the Micro-TENN, in some cases the cortical neurons infiltrated slightly deeper into the fibrin Micro-TENN and formed numerous small clusters. Extending neurites produced networks between these clusters and also penetrated farther into the Micro-TENN interior (FIG. 3B-FIG. 3E). In other cases, the desired architecture of the cortical neuron in the collagen+laminin Micro-TENNs was achieved, consisting of a dense primary cluster on neurons with axonal projections through the Micro-TENN central canal (FIG. 3F).

### Micro-TENN Implantation

Living Micro-TENNs were stereotaxically injected to connect thalamic structures with the cortex in rats. At terminal time-points, rats were transcardially perfused with fixative and the brains processed for immunohistochemical analysis of implant cell survival, maintenance of cytoarchitecture, and integration.

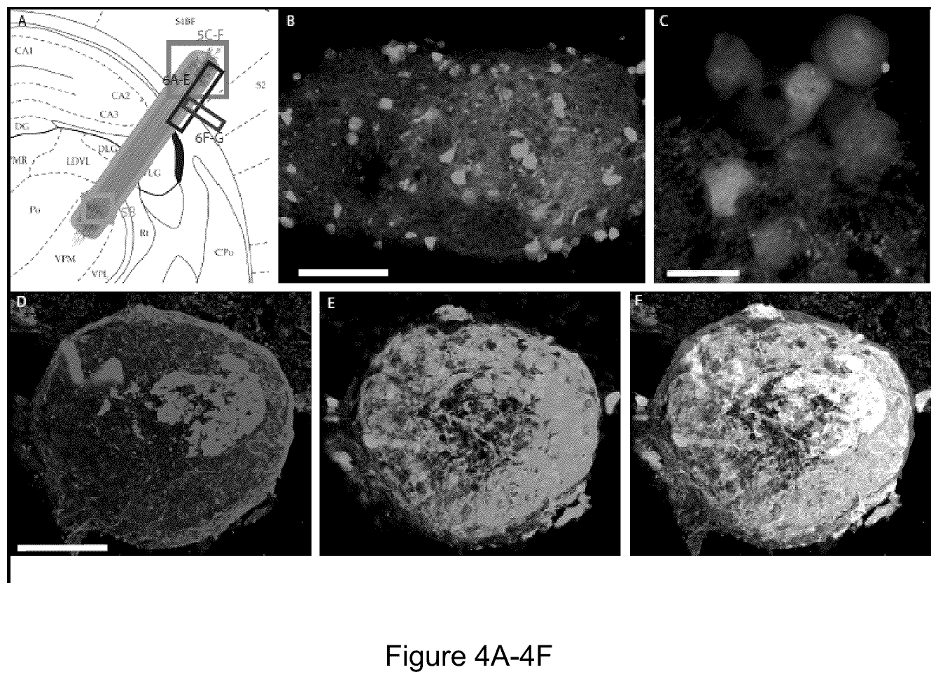

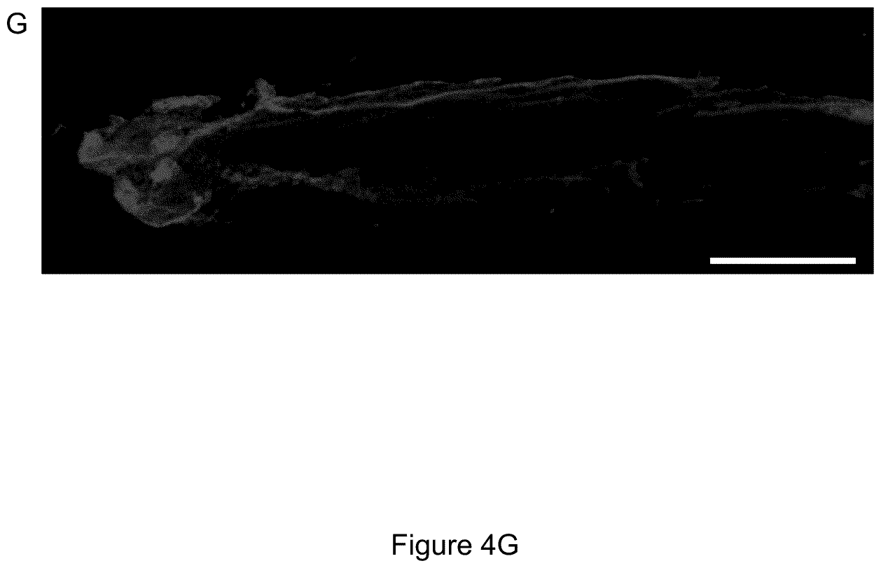

Cortical Micro-TENNs were implanted and acutely harvested. At 1 week post implant, surviving neurons were found in 67% of the cortical Micro-TENNs (FIG. 4B). At 1 month post-implant, surviving clusters of GFP+neurons were found in 50% of the Micro-TENNs near the cortical surface. Immunohistochemistry verified that these dense clusters within the Micro-TENN ends indeed contained living neurons, as indicated via MAP-2 labeling for neuronal somata and dendrites as well as neurofilament labeling for axons (FIG. 4C-F). DRG Micro-TENNs were delivered into the cortex and harvested at 3 days post implant. Surviving neurons were found in the Micro-TENN interior. They remained in a tight cluster with their axons projecting parallel to the cortico-thalamic pathways, thus demonstrating maintenance of cytoarchitecture (FIG. 4G).

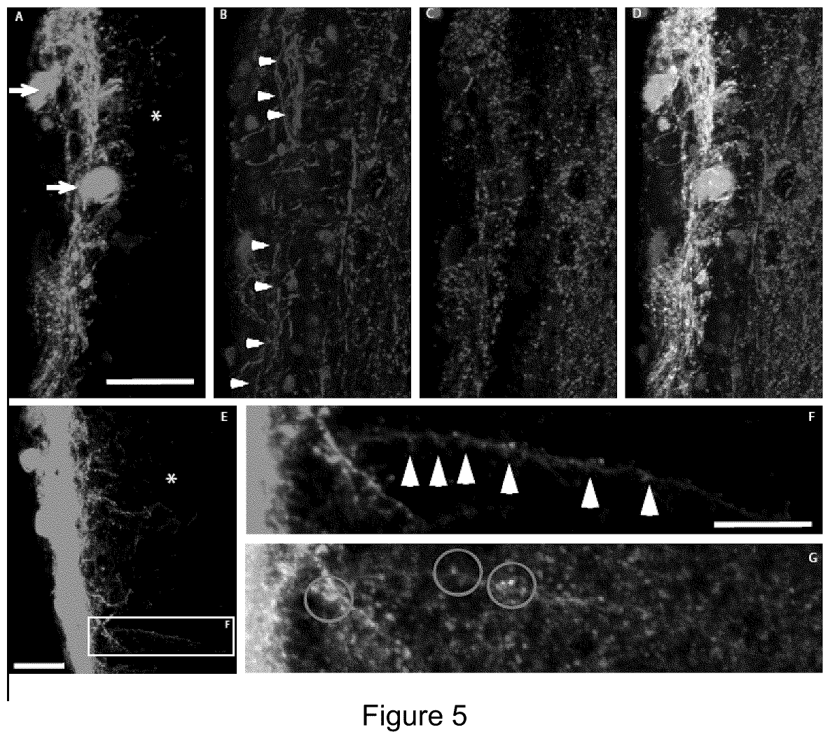

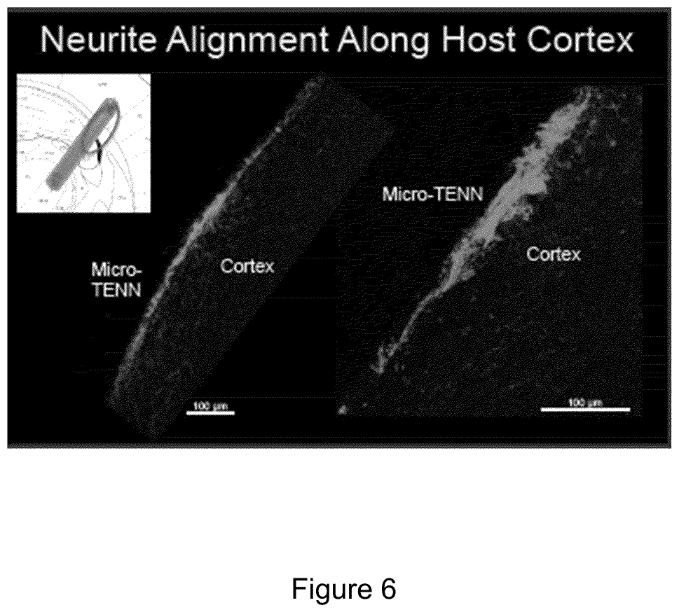

In addition, deeper along the length of the Micro-TENNs, implanted neurons and neurites were observed to be aligned longitudinally at the Micro-TENN-cortex interface and running along the cortical-thalamic axis (FIG. 5A-D). As the agarose in the Micro-TENNs gradually broke down, the neurons appeared to have shifted laterally to this interface. Immunohistochemistry revealed that these longitudinal Micro-TENN projections consisted of implanted neuronal somata (as denoted by arrows) as well as aligned axons (as shown by arrowheads). Synapsin-positive puncta was also observed, suggesting synaptic inputs along these projections. Neurite alignment is also seen in FIG. 6, in which surviving neurons within the Micro-TENN were observed aligned to the cortical thalamic axis. This demonstrates that Micro-TENN neurons and neurites aligned radially along the Micro-TENN—cortex interface.

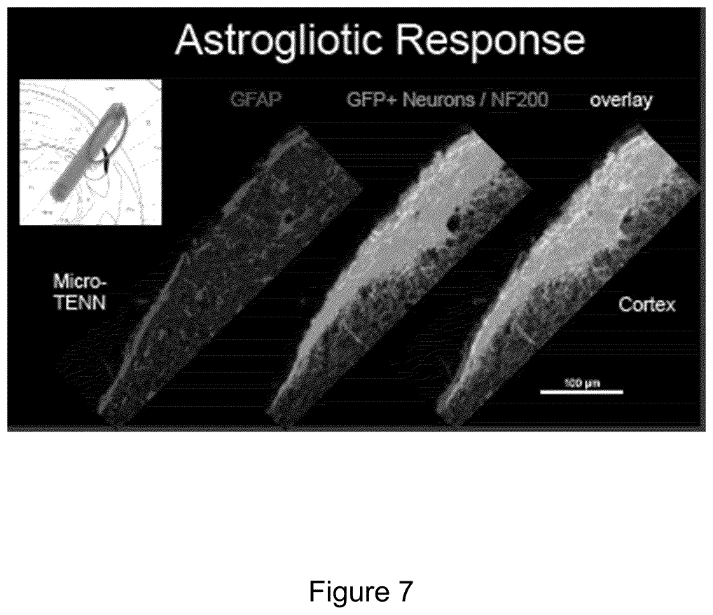

Interestingly, these implanted neurons and axonal tracts seemed uninhibited by host reactive astrocytes, as shown through GFAP labeling for reactive astrocytes. This demonstrates that Micro-TENN neurons migrated through the glial scar to integrate with the cortex (FIG. 7).

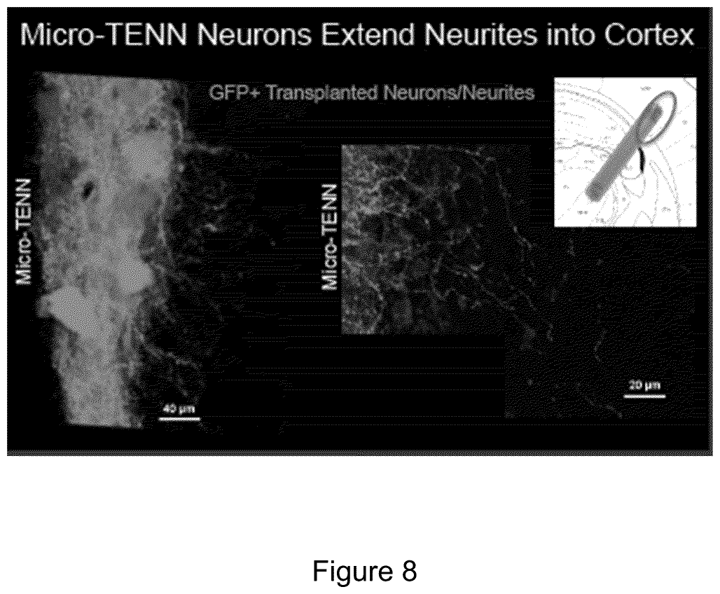

Additionally, it was found that Micro-TENN neurons extended neurites into the host cortex (FIG. 5E). A higher magnification image shows neurites from the Micro-TENN penetrating deep into the host cortex, in some cases up to several hundred microns (FIG. 8). Immunohistochemistry has also suggested that these neurites—from the implanted neurons-formed synapses with host cortical neurons. In particular, putative dendritic spines along ingrowing neurites were morphologically identified and synapsin-positive puncta in immediate proximity to these dendritic spines were found (FIG. 5F-G).

Collectively, these results exhibit the successful translation of the Micro-TENN technology for use with primary cortical neurons. They also demonstrate the ability of the Micro-TENNs to survive long-term in vivo, as well as their capacity to extend processes into the host tissue.

### Micro-TENNs

Although axonal loss is a prominent feature of many neurological diseases and trauma, there is currently no strategy capable of repairing long-distance axonal connections in the brain. As presented herein, micro-tissue engineered neural networks containing neurons and long axon tracts were developed to facilitate nervous system repair by directly restoring lost neural circuitry. These constructs can thus be utilized to promote functional regeneration following injury to the brain.

Several different techniques have previously been used to grow neural cells in specific 2D and 3D geometries, i.e., cell patterning. Removable silicon barriers have been applied to culture surfaces in order to isolate neural populations during plating. The barriers were then removed to enable outgrowth and create distinct somatic and axonal regions (Smith et al., 1999, J Neurosci 19 (11): 4263-4269). Likewise, desired patterns of growth have been created by selectively treating the culture surface with cell adhesive factors and proteins. The methods most commonly used to selectively treat surfaces in 2D include micro-stamping, photolithography, and micro-fluidic deposition. Micro-stamping involves loading an etched polymer stamp with adhesion molecules and bringing it into contact with the culture substrate (Nam and Wheeler, 2004, Conf Proc IEEE Eng Med Biol Soc. 6:4049-52; Wheeler et al., 2004 Conf Proc IEEE Eng Med Biol Soc 7:5337-5339). Photolithography uses light exposure through a mask followed by chemical treatment to immobilize proteins in precise geometries (Sorribas et al., 2002, Biomaterials, 23 (3): 893-900). Lastly, microfluidic systems control the flow of small amounts of liquids in order to create gradients of signaling molecules (Millet et al., 2010, Lab Chip, 10 (12): 1525-1535). This microfluidic technology has recently been advanced to produce 3D multi-layered scaffolds for neural cell culture (Kunze et al., 2011, Biomaterials, 32 (8): 2088-2098). Kunze et al. used their microfluidic device to generate a 3D scaffold with two cell-hydrogel layers separated by two cell-free hydrogel layers. Furthermore, they differentially loaded the hydrogel layers with B-27 and found this impacted both the neurite length and density. Other methods to direct outgrowth in 3D include the use of structured scaffolds to provide guidance cues to neurities (Yoo et al., 2011, Exp Neurobiol 20 (2): 110-115). For example, Yoo et al. developed a micromesh that created patterned neurite growth along the mesh's nylon fibers.

In order to recreate the neuroanatomical features of axonal tracts, a novel method was devised to create distinct somatic and axonal regions. Specifically, the ability of various hydrogel densities to control the movement of the cell bodies was explored. Coupled with precise cell delivery, the optimized composition and density of the Micro-TENN core produced the ideal Micro-TENN cytoarchitecture: neuronal ganglia on the Micro-TENN extremes with an interior comprised exclusively of neurites.

As described herein, bi-directional DRG Micro-TENN constructs were successfully developed, in which neuronal populations were plated on both ends. With the growth of long axonal tracts between the two populations, the bi-directional Micro-TENNs formed the basic neural building blocks to repair neural circuitry. Typical bi-directional cytoarchitecture comprised DRG ganglia located at the Micro-TENN ends, with axonal projections penetrating through the Micro-TENN interior. The length of neurite ingrowth was measured at weekly time points in order to quantify the rate of growth of the Micro-TENNs. It was found that, on average, they grew approximately 0.16 mm/day over the course of 6 weeks. By 42 DIV, it was frequently observed that axons from the two populations had overlapped in order to form constructs measuring approximately 2 cm. This is believed to be the longest recorded instance of dorsal root ganglia outgrowth in a 3D scaffold in vitro. Attempts to make the Micro-TENNs longer than 2 cm resulted in reduced axonal penetration, which suggests that soluble factors released from the target cells and axons are critical to maintaining axon growth. It is possible that these factors can only diffuse in sufficient concentrations in constructs shorter than 2 cm. Thus, in order to generate constructs longer than 2 cm, exogenous chemotaxic/neurotrophic support may need to be incorporated into the Micro-TENNs.

Bi-directional Micro-TENNs utilizing primary cerebral cortical neurons were also developed. Although collagen alone has previously been shown to support cortical neurons (O'Connor et al., 2001, Neurosci Lett, 304 (3): 189-193; Woerly et al., 1996, Neurosci Lett, 205 (3): 197-201), it was found that fibrin or collagen+laminin supported more robust cortical health and axonal extension than collagen inside the Micro-TENNs. In contrast to the DRG Micro-TENNs, in some cases the cortical neurons infiltrated the Micro-TENN core rather than forming distinct ganglia on the Micro-TENN ends. However, with improved ECM constituents and delivery, the desired architecture of discrete neuronal populations and axonal tracts was achieved.

Neural cell implantation has been investigated as a potential treatment for neurodegenerative disorders, such as Huntington's disease and Parkinson's disease, as well as for stroke. Previous studies have typically involved the implantation of fetal grafts, single cell suspensions, or cells in 3D matrices (Denham et al., 2012, Front Cell Neurosci 6:11; Fawcett et al., 1995, Exp Brain Res, 106 (2): 275-282; Mine et al., 2013, Neurobiol Dis 52:191-203; Ren et al., 2013, Int J Clin Exp Pathol 6 (2): 230-241; Sinclair et al., 1999, Eur J Neurosci 11 (12): 4341-4348; Yoo et al., 2011, Exp Neurobiol 20 (2): 110-115; Tate et al., 2002, Cell Transplant 11 (3): 283-295; Tate et al., 2009 J Tissue Eng Regen Med 3 (3): 208-217). The delivery method for these procedures is relatively non-invasive, as it involves the injection of only a few microliters of suspended cells or an ECM hydrogel using a microsyringe. The studies have found varying implant attrition rates; in some cases, evidence of implant integration with the host tissue has been found. Retrograde tract tracing has been used to confirm the establishment of implant projections to appropriate targets (Mine et al., 2013, Neurobiol Dis 52:191-203). Another outcome assay used to affirm implant integration is immunohistochemical labeling for synaptophysin or synapsin (Qiu et al., 2005, Exp Eye Res, 80 (4): 515-525). Lastly, electrophysiological recordings have been used to test the ability of implanted neurons to fire action potentials and function properly in an integrated neural system (Denham et al., 2012, Front Cell Neurosci 6:11).

The present implant differs significantly from previous approaches in that the cytoarchitecture of the Micro-TENN is created in vitro and implanted en masse. In order to assess Micro-TENN cell survival, maintenance of cytoarchitecture, and integration following delivery into the brain, Micro-TENNs were stereotaxically injected to connect thalamic structures with the cortex in rats. At acute time points, cell survival, as well as maintenance of cytoarchitecture, was observed in both DRG and cortical Micro-TENNs. For the chronic Micro-TENN implant study, cortical Micro-TENNs were studied exclusively. While not wishing to be bound by any particular theory, it was thought that these cortical Micro-TENNs would have a greater chance of integrating with the surrounding host cells in the brain due to the intrinsic ability of cortical neurons to assimilate into the neural cytoarchitecture and synapse with both cortical and thalamic neurons. Remarkably, at 1 month post-implant, evidence of Micro-TENN cell survival and structural integration with the host cortex was found. Specifically, putative dendritic spines were observed along the ingrowing neurites from the implant cells, and synapsin-positive puncta were found in immediate proximity to these dendritic spines. This suggests that host axons terminated in synapsin-positive presynaptic specializations to synapse with ingrowing dendrites from implanted neurons. Possibly, these inputs could be relayed down the axonal projections previously shown that run longitudinally along the cortical-thalamic axis.

Based on the data described herein, these unique Micro-TENNs comprising neurons and long axonal tracts will be useful to reconstitute lost neuroanatomy, successfully integrate with host neural circuitry, and re-establish lost thalamocortical tracts to facilitate functional recovery.

Described herein is the development of micro-tissue engineered neural networks, comprising pre-formed implantable constructs that recapitulate the basic neural systems level functional unit: discrete groupings of neurons spanned by long axonal tracts. The Micro-TENNs described herein facilitate nervous system repair by simultaneously replacing lost neurons and restoring long-distance axonal connections in the brain. These findings described herein demonstrate long term in vivo survival of the Micro-TENNs, as well as their ability to integrate with the host tissue.

### Example 2: Optimization of Micro-TENN Construct

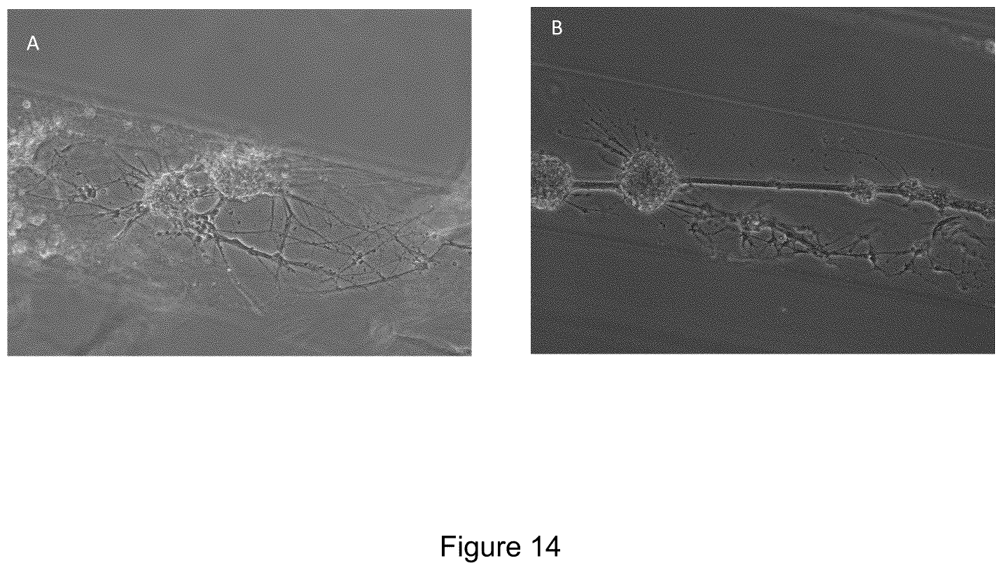

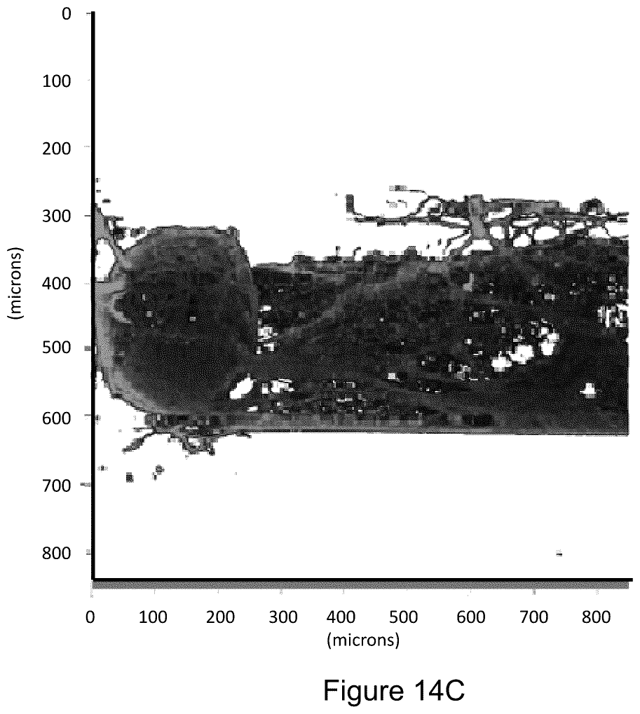

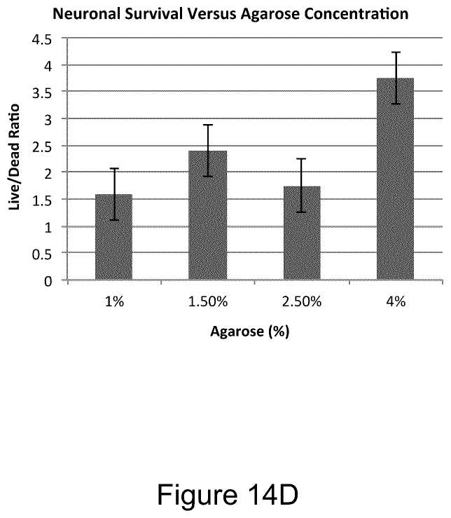

Techniques were developed to engineer micron-scale three-dimensional (3-D) tubular hydrogel constructs. The constructs comprise hollow agarose tubes (125-800 micron inner diameter×500-1000 micron outer diameter) up to several centimeters long. The agarose varied in concentration between 0.5-4.0%. Techniques have been developed to fill the constructs with a bioactive extracellular matrix (ECM)-fibrin, collagen (I and IV), laminin, hyaluronic acid, and combinations thereof. The construct biomaterial(s) and construction has been optimized for neuronal survival and axonal outgrowth. Here, it was found that increasing agarose concentration improved survival and neurite outgrowth of cerebral cortical neurons (maximum survival was found at 4% agarose) (FIG. 14). It was found that procedural modifications are often necessary on a per-ECM basis, and each neuronal subtype and density requires a re-optimization of ECM constituents and concentration. For instance collagen I ECM was sufficient for DRG neuronal survival and outgrowth, but cerebral cortical neurons required collagen I+laminin and/or fibrin ECM for survival and growth. In particular, cortical neuronal survival increased with increasing laminin concentration up to 1 mg/mL (mixed with collagen to provide 3D support). These growth conditions—optimal hydrogel concentration and ECM composition—have been optimized to allow for bidirectional as well as longer axonal growth. The effects of adding soluble factors such as nerve growth factor (NGF) and brain-derived neurotrophic factor (BDNF) on neuron health and axon elongation within constructs are investigated. For example, developing Micro-TENNs with gradients of growth factors could potentially exceed axon extension capabilities achieved by ECM treatments alone, and therefore will allow generation of longer Micro-TENNs. Moreover, maximizing survival and neurite outgrowth in vitro will ultimately improve survival and synaptic integration in vivo, and is thus crucial for the ultimate function of Micro-TENNs to restore brain pathways.

The creation of bi-directional Micro-TENNs (FIG. 10) permitted a 4-fold increase in the length of axon extension attainable in the micro-conduits versus unidirectional. Overall, the survival of low densities of neurons (i.e. relatively few cells), precise delivery and stability in precise locations, and targeted long-distance axonal extension in a 3-D microenvironment are unprecedented. Of note, cerebral cortical neurons are particularly challenging to grow even under simple, 2-D conditions-survival and growth of these neurons in miniature 3-D tubes is a significant development, and required the identification of unique bioactive ECM combinations versus those supporting DRG neurons.

### Example 3: Assessment of Micro-TENN Integration and Functional Recovery

Integration of implanted Micro-TENNs and the functional recovery of subjects with implanted Micro-TENNs are examined using a rodent model by forming a lesion in the well-defined whisker-sensory pathway causing pain-like whisker hypersensitivity. Following characterization of this response, tailored Micro-TENNs are delivered to reconstruct the lost tract with functional and structural outcomes as described elsewhere herein.

The experiments presented herein focus on the well-studied rat whisker system by altering a secondary whisker pathway to induce chronic pain/hypersensitivity in rats. Based on previous research, the lesion to the paralemniscal pathway should cause the response to become over-excitatory, thereby resulting in pain/hypersensitivity due to whisker stimulation. Previous research has indicated that normal rat whisker behavior to explore their surroundings can take on a pathological condition representing pain or hypersensitivity (McNamara et al., 2010, J Neurotrauma, 27 (4): 695-706). After injury, rats shifted from exploration to withdrawal from whisker stimulation. The behavior was repeatable and well aligned with injuries over several studies (McNamara et al., 2010, J Neurotrauma, 27 (4): 695-706; Learoyd et al., 2012, Behav Brain Res, 226 (1): 197-204). Subsequent research indicated that the switch in behavior was due to increased excitatory signaling in the ventral posteromedial thalamic nucleus (VPM) in the primary whisker sensory pathway (lemniscal) (Thomas et al., J Neurotrauma, 2012, 29 (2): 187-200). Since the VPM has a direct excitatory connection to the somatosensory cortex, the research suggests that an increase in excitatory signaling results in the switch from whisker perception to pain. To recreate this pain/hypersensitivity published alterations in secondary whisker sensory systems was reviewed (Diamond et al., Nat Rev Neurosci, 2008, 9 (8): 601-612). The work revealed that the Zona Incerta (ZI) has an inhibitory input onto the rat sensory circuitry that is controlled by a descending pathway from the motor cortex (Lavallee, 2005, J Neurosci, 25 (33): 7489-7498). Reduced activity in the ZI led to an increase in neural firing of the posterior thalamic nucleus (Po) and rat somatosensory barrel cortex (Masri et al., 2009, J Neurophysiol, 102 (1): 181-191). The research indicates that an interruption in the motor cortex pathway feeding the ZI could result in pain/hypersensitivity (Thomas et al., J Neurotrauma, 2012, 29 (2): 187-200). Therefore, the cortex to ZI pathway becomes a potential target to emulate pain/hypersensitivity.

The lesion and implant is performed as described elsewhere herein. After the lesion, behavioral testing of the animals is performed. Behavior is tested via the Whisker Nuisance Test (WNT) (McNamara et al., 2010, J Neurotrauma, 27 (4): 695-706; Learoyd et al., 2012, Behav Brain Res, 226 (1): 197-204). The WNT consists of physical whisker stimulation for three 5 min periods. Blinded scorers will conduct the WNT by judging 19 different exhibited behaviors (0, absent, 1, present, 2, profound). Previous research has found this method to be a sensitive indicator of sensitivity/pain. Based on previous research, there are 10 animals per group. The groups consist of (1) unlesioned; (2) lesioned+Micro-TENN; and (3) lesioned without Micro-TENN. Animals are tested for behavior beginning at 7 days and weekly thereafter. Before animal sacrifice at 1 month and 3 months, electrophysiology is performed.

Fiber tract integration with the barrel cortex is assessed using whisker stimulation during an anesthetized preparation. Whiskers associated with the lesioned region are stimulated with piezo-electric stimulators at known stimulus frequencies (0.5-10 hz) (Melzer et al., 2006, J Neuorsci, 26 (47): 12198-12205). Recordings of the neuronal output are obtained via tetrodes (Neuronexus) located in the barrel fields via a Neuralynx acquisition system. Responses quantify the reconnection of the disrupted pathway. Following electrophysiological testing, histology is performed. Additionally, with a subset of brains, diffusion tensor imaging (DTI) is used employing a 3-D multi-spin echo protocol with oval sampling in the two-phase encoding directions (Kim et al., 2012, NMR in Biomedicine, 25 (1): 104-112; Kim et al., 2009, NMR in Biomedicine, 22 (3): 303-309). This protocol is capable of achieving an acquired isotropic resolution of 64 microns in rodent specimens.

The focused lesion increases the nociceptive behavior and whisker pain in the lesioned animals. Lesioning is confirmed by histology, and little intrinsic regeneration occurs. Micro-TENNs restore this pathway in a two-stage mechanism: forming synapses with the Micro-TENN neurons (at both ends of the Micro-TENN) and then using the Micro-TENN axons as a new relay across the neuroanatomical structures. Histological and electrophysiological outcomes mirror the improvements in behavior. Overall, a notable moderate restoration of function is observed with the use of Micro-TENNs.

### Example 4: Transiently Rigid Outer Shell of Micro-TENN to Allow for Needle-Less Delivery

Described herein is the development of a biomaterial strategy to allow for “needle-less” delivery of the Micro-TENN construct, meaning the Micro-TENN itself has sufficient mechanical strength to penetrate the brain without using a rigid needle (i.e. one made of metal or plastic). Moreover, this biomaterial strategy was designed such that once in the brain environment, the material softens to match the mechanical properties of the brain, and thus will not cause unnecessary neuroinflammation and gliosis due to chronic mechanical mismatch between the construct and the surrounding brain tissue, for example as seen for rigid electrodes in the brain.

Experiments were conducted in order to develop a strategy which minimizes implantation damage to native brain tissue while protecting Micro-TENN neurons during implant. The design objectives were to (1) minimize footprint of implant and inserter into brain; (2) ensure cell survival and viability; and (3) penetrate the brain parenchyma but then soften to match material properties of brain.

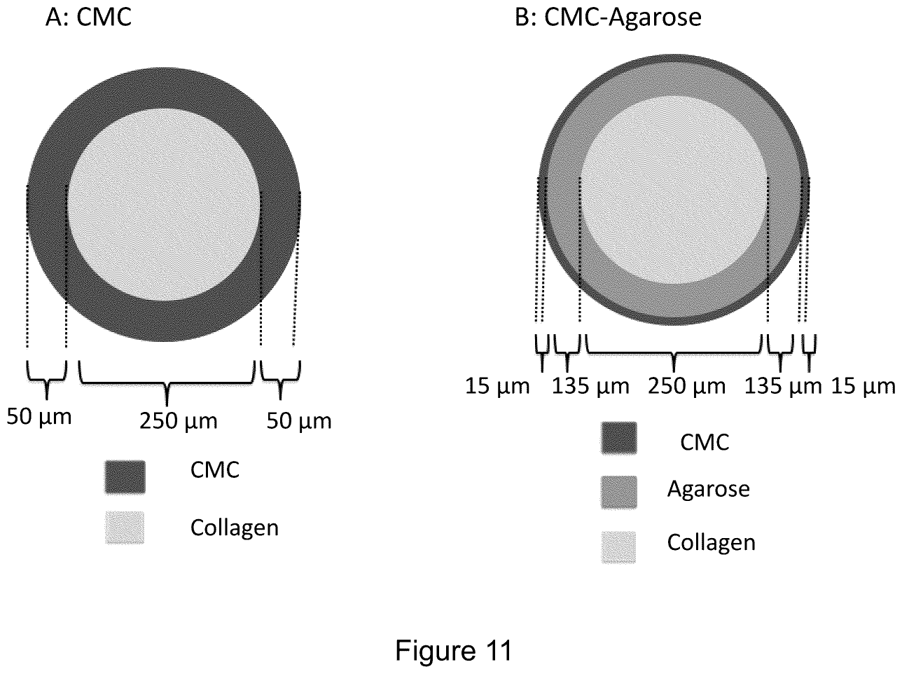

Two different strategies were examined. One strategy was to use a different material for the Micro-TENN itself (FIG. 11A). A second strategy was to use a hard outer coating on the existing Micro-TENN (FIG. 11B). This new material must provide stiffness for insertion that protects Micro-TENN neurons from manipulation damage. After insertion, the coating dissolves and/or is chemically modified to maximize Micro-TENN integration.

The use of multiple biomaterials was explored for this specific application, knowing that the material needed to have controlled and modifiable physical properties, be stable over the period of culture and/or implantation, and be biocompatible and non-inflammatory. Following initial experiments it was decided to pursue “Design B” involving the outer coating on our existing agarose-based construct. Also, it was decided to that the outer coating was to comprise carboxymethylcellulose (CMC). CMC is a cellulose derivative with carboxymethyl groups bound to hydroxyl groups. The functional properties depend on degree of substitution of cellulose structure and degree of polymerization. CMC possesses unique properties in that it is stiff in a dehydrated state and gel-like hydrated state, with a short transition period between states at micro-dimensions. Also, CMC is nontoxic to humans and animals, inexpensive, and widely available.

CMC-coated Micro-TENNs were created using multiple fabrication methods including first making 20% low viscosity CMC and then painting/applying, dipping, and rolling the agarose construct. However, the optimal protocol involved first applying the CMC powder to a fully formed agarose construct and then hydrating it in place—this design resulted in the most uniform and consistent CMC coating on the construct. Moreover, multiple thicknesses have been utilized and the dehydration time has been optimized to manipulate the mechanical properties of the CMC coating.

### CMC-Coated Construct Fabrication

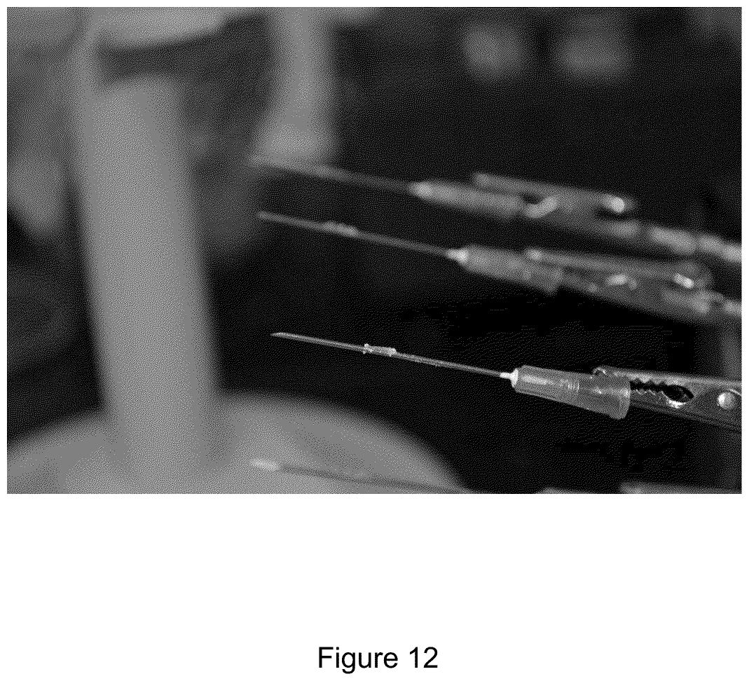

After the tubular agarose construct has been fabricated, the construct was coated in carboxymethylcellulose (CMC). The dry low viscosity CMC was spread on a glass slide, approximately slightly larger than the length and width of the construct with height of about twice the construct. The construct was then placed on the tip of a stainless steel needle (FIG. 12) and visually inspected to be straight (manually adjusted via another needle). The needle with construct was then gently patted onto the dry CMC. With the addition of luL of liquid (PBS, water, or other media) and the native liquid in the construct, the CMC became moistened to create a uniform coating around the construct. The CMC-coated construct was then left to dry for 10-15 minutes on the needle tip. The coated construct was easily separated from the needle when dry.

### CMC-Coated Construct Insertion

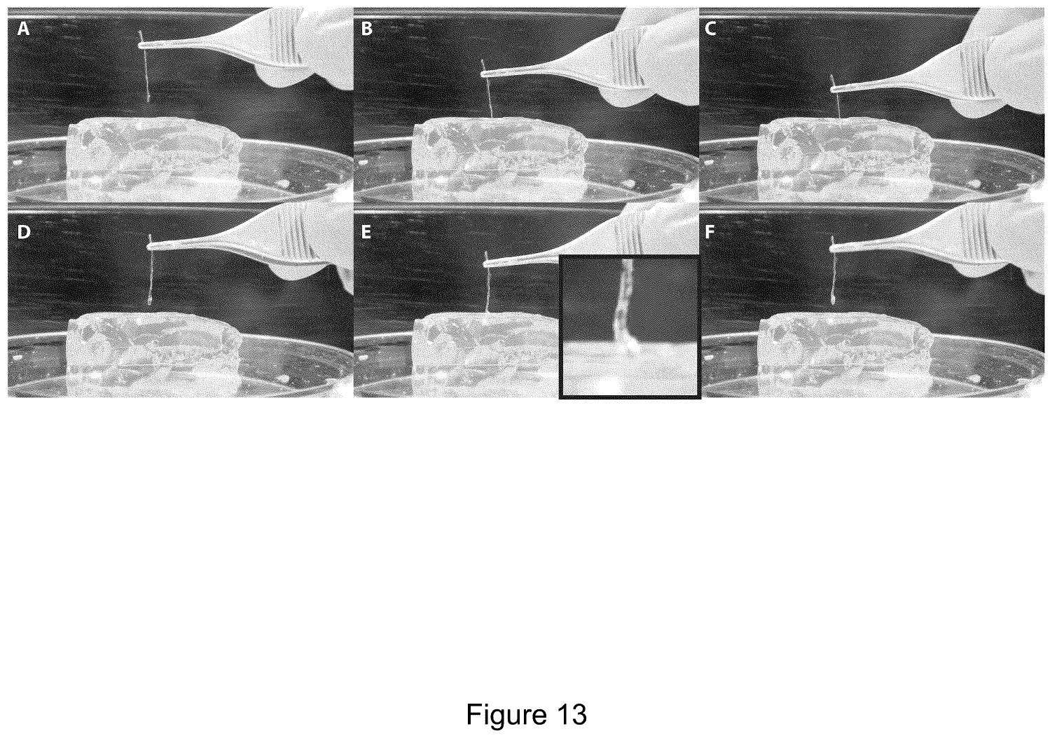

For insertion, the CMC-coated construct was loaded into the tip of an unbeveled needle. While the CMC-coated construct is mostly straight, the small undulations in the implant allows for the construct to stay within the needle tip. A plunger (another smaller unbeveled needle sealed with bonewax) was positioned into the rear of the loaded needle. When placed above the cortex of the animal the plunger was pushed through the CMC-construct loaded needle to inject the CMC-coated construct into the cortex. FIG. 13 depicts the exemplary insertion of the coated construct into a surrogate brain like material. As seen in FIG. 13C, the coated construct can be inserted directly into the surrogate. After about 5 seconds, the construct softens. This softening is visualized by the fact that after the construct is removed from the surrogate, it loses the ability to penetrate the surrogate (FIG. 13E).

### Example 5: Detailed Concept

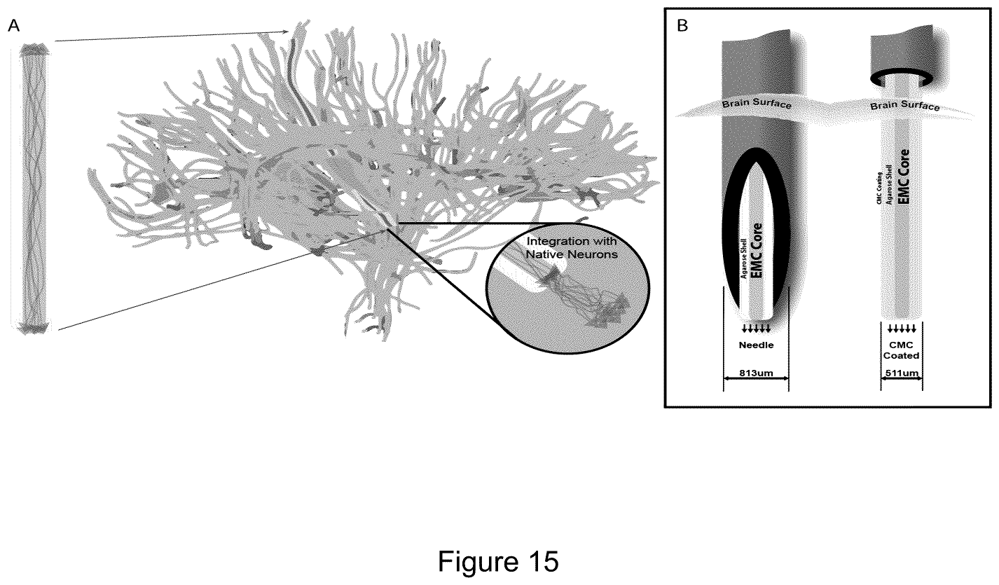

The detailed concept is described in FIG. 15, which illustrates Micro-TENN integration and the potential benefits of needle-less delivery (smaller and thus less invasive). The results described herein were generated via the same experimental procedures described herein. FIG. 15, comprising FIG. 15A and FIG. 15B, depicts the Micro-TENN concept. FIG. 15A depicts a tissue-engineering construct (Micro-TENN) with discreet populations of cells at either end, connected via long-distance axonal connections. Micro-TENNs can be used to repair long-distance axonal pathways, depicted in tractography recreation of pathways in brain (FIG. 15A, right). The Micro-TENN construct emulates axonal pathways with neuronal clusters at ends to allow for reintegration with native neurons (FIG. 15A, right, inset). FIG. 15B depicts needle versus needle-less insertion of Micro-TENNs into brain. The Needle method (FIG. 15B, left) uses a needle and plunger system to pierce the brain surface and then inject Micro-TENN into brain parenchyma. The Needle-less method (FIG. 15B, right) uses CMC-coated Micro-TENN where the needle is brought to the surface of the brain, and coated Micro-TENN penetrates the brain surface and ultimately enters the brain parenchyma.

### Example 6: Method of Making Micro-TENN

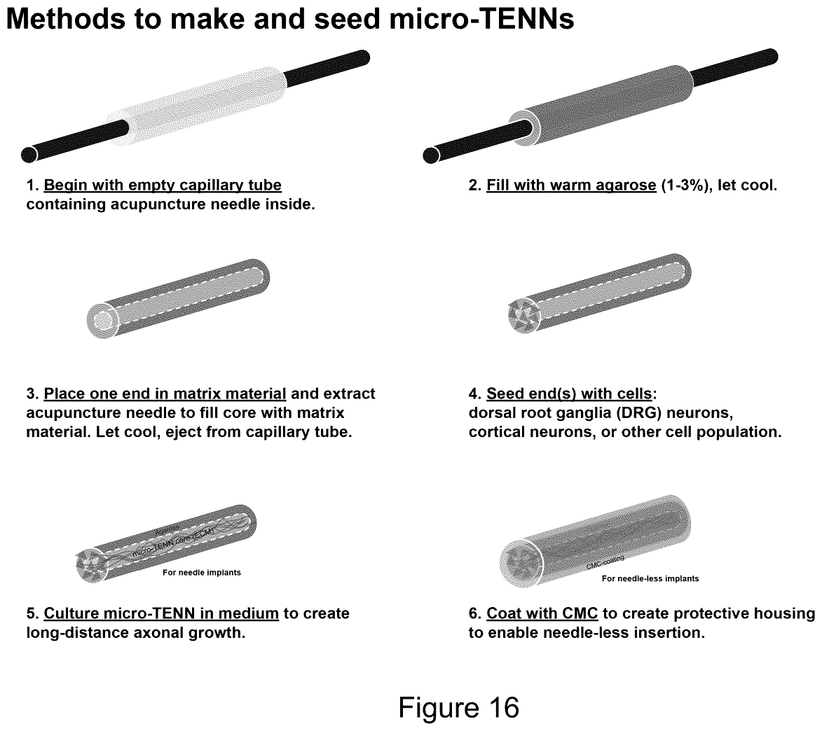

The method of making Micro-TENN is illustrated in FIG. 16. (A) starting with an empty capillary tube with an acupuncture needle through the center; (B) one end of the capillary tube is placed in warm agarose. Capillary action draws up the agarose (the warmer the agarose solution, the more complete the draw). Note, the end of the acupuncture needle is generally flush with the end of the capillary tube when the agarose is drawn up; (C) after the agarose has cooled, one end of the construct is placed in the matrix material that will be used to fill the core of the microconduit. The acupuncture needle is drawn up slowly from the opposite end, careful holding the agarose so as to keep it within the capillary tube. After cooling, the agarose tube with filled inner core is ejected by inserting a needle or other plunger in one end; (D) the ends of the microconduit can be cut at a 45 degree angle (w.r.t. to plate bottom) in order to create a small trough for cells to be seeded. Seeded microconduits can be anchored in media via addition agarose struts or other methods; (E) after several days in vitro, neurons will exhibit robust features and axonal outgrowth; and (F) when the Micro-TENN has matured to the level desired, Micro-TENNs can be coated with the CMC-coating.

### Example 7: Advancements in Cytoarchitecture and Maturation of the Micro-TENNs

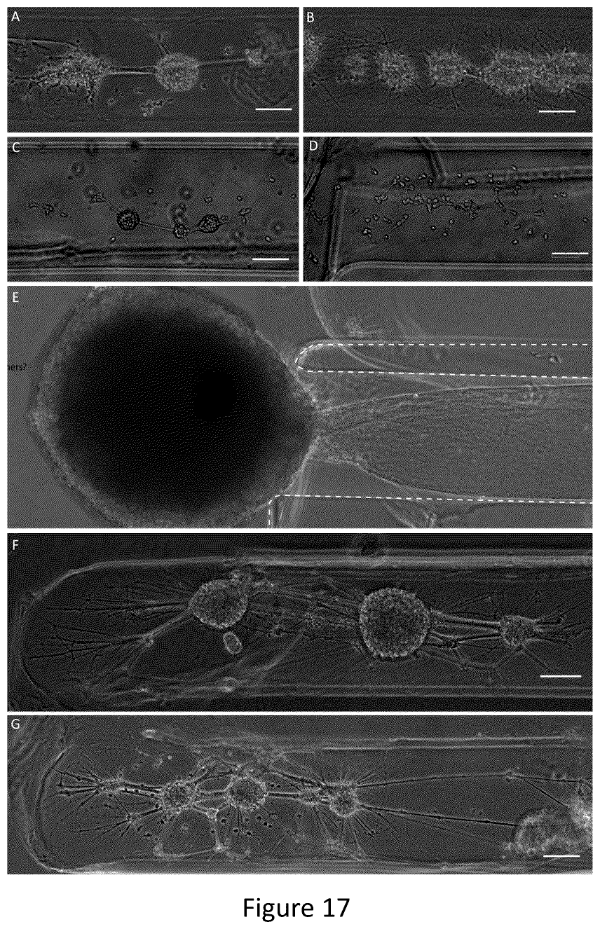

The FIG. 17A depicts representative cortical neuronal Micro-TENNs (750 μm OD, 350 μm ID; 3% agarose shell; 1 mg/ml collagen IV+1 mg/ml laminin core; 56,000 cells/ml) at 4 DIV. FIG. 17B depicts representative cortical neuronal Micro-TENNs (750 μm OD, 350 μm ID; 3% agarose shell; 1 mg/ml collagen IV+1 mg/ml laminin core; 72,000 cells/ml) at 4 DIV. FIG. 17C depicts representative cortical neuronal Micro-TENNs (750 μm OD, 350 μm ID; 3% agarose shell; 1 mg/ml collagen IV+1 mg/ml laminin core; 36,000 cells/ml) at 5 DIV. The primary cortical neurons were resuspended in Neurobasal media before plating. FIG. 17D depicts representative cortical neuronal Micro-TENNs (750 μm OD, 350 μm ID; 3% agarose shell; 1 mg/ml collagen IV+1 mg/ml laminin core; 36,000 cells/ml) at 5 DIV. The primary cortical neurons were resuspended in extracellular matrix solution (1 mg/ml collagen IV+1 mg/ml laminin) before plating. FIG. 17E depicts representative cortical neuronal Micro-TENNs (750 μm OD, 350 μm ID; 3% agarose shell; 1 mg/ml collagen IV+1 mg/ml laminin core; 36,000 cells/ml) at 7 DIV. FIG. 17F depicts representative cortical neuronal Micro-TENNs (750 μm OD, 350 μm ID; 3% agarose shell; 1 mg/ml collagen IV+1 mg/ml laminin core; 36,000 cells/ml) at 8 DIV. FIG. 17G depicts representative cortical neuronal Micro-TENNs (750 μm OD, 350 μm ID; 3% agarose shell; 1 mg/ml collagen IV+1 mg/ml laminin core; 36,000 cells/ml) at 18. Scale bars=100 μm.

Early in development, Micro-TENNs were plated at high densities: 56,000 cells/ml (A) and 72,000 cells/ml (B), but cytoarchitecture was optimal when a lower density of 36,000 cells/ml was used (C-F). Resuspending primary cortical neurons in Neurobasal media before plating (C) produced more consistent and optimal cytoarchitecture than resuspending in extracellular matrix solution (1 mg/ml collagen IV+1 mg/ml laminin) before plating (D). While cortical cultures tend to invade the interior channel of the Micro-TENN, an alternative architecture can occur where a cluster of cells remains external to Micro-TENN (E, dotted line=agarose walls of Micro-TENN). The optimal cytoarchitecture was maintained up to 21 DIV by plating at low cell density, 36k, in Neurobasal media. Representative examples of this at 8 DIV (F) and 18 DIV (G) are shown here. Scale bars=100 μm.

### Example 8: Verification of Neuronal and Axonal Phenotype in “Mature” Micro-TENNs

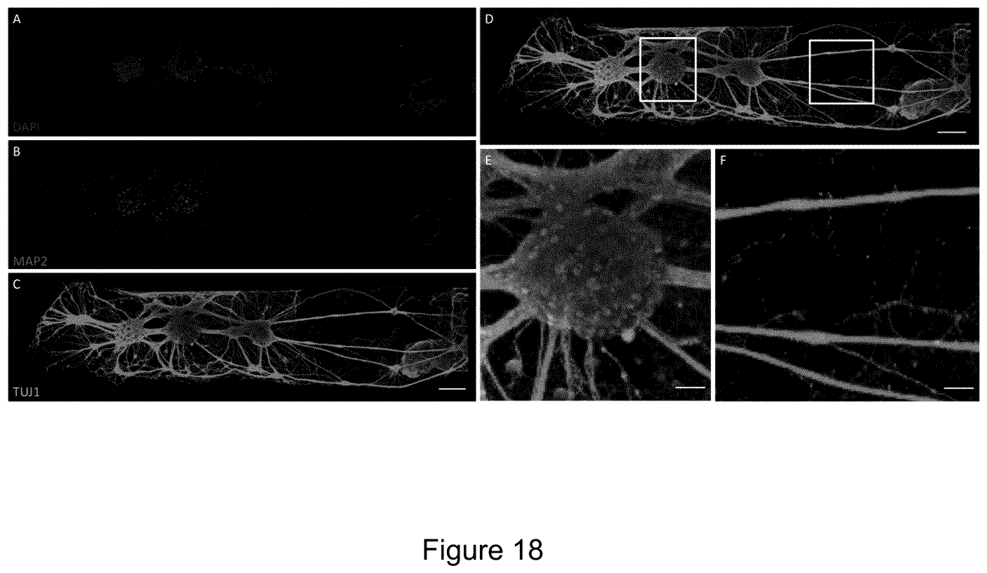

The long-term cellular phenotype and Micro-TENN architecture are depicted in FIG. 18. FIG. 18A depicts DAPI stained representative cortical neuronal Micro-TENN (750 μm OD, 350 μm ID; 3% agarose shell; 1 mg/ml collagen IV, 1 mg/ml laminin core; 36,000 cells/ml) fixed at 18 DIV. Scale bars=100 μm. FIG. 18B depicts MAP2 stained representative cortical neuronal Micro-TENN (750 μm OD, 350 μm ID; 3% agarose shell; 1 mg/ml collagen IV, 1 mg/ml laminin core; 36,000 cells/ml) fixed at 18 DIV. Scale bars=100 μm. FIG. 18C depicts TUJ1 stained representative cortical neuronal Micro-TENN (750 μm OD, 350 μm ID; 3% agarose shell; 1 mg/ml collagen IV, 1 mg/ml laminin core; 36,000 cells/ml) fixed at 18 DIV. Scale bars=100 μm. FIGS. 18A-D depict 3D-projection of Micro-TENN from channel opening (left of image) to mid-tube (right of image) at 10×. Scale bars=100 μm. FIG. 18E depicts representative nucleus of MAP2-positive cells projecting axonal tracts in multiple directions at 40×. Scale bars=25 μm. FIG. 18F depicts laterally projecting TUJ1-positive axonal tracts at 40×. Scale bars=25 μm.

### Example 9: Needle-Less Brain Penetration

CMC-coated Micro-TENNs and steel wires were mounted on a custom made pin vice holder mounted to a 5N load cell on an Instron 5542 mechanical tester. Mildly-dehydrated, CMC-coated Micro-TENN (diameter: 511.40 μm+/−29.24 μm) and stainless steel wire (508 μm) were lowered into a block of 0.6% agarose (similar stiffness to brain) at 2 mm/s while the required insertion force was measured. This revealed that at the specified diameters, the needed insertion force was 22 mN. Moreover, CMC-coated Micro-TENNs withstood 893 mN+/−457 mN, demonstrating that the CMC-coated Micro-TENNs were strong enough to be implanted needle-lessly into the brain.

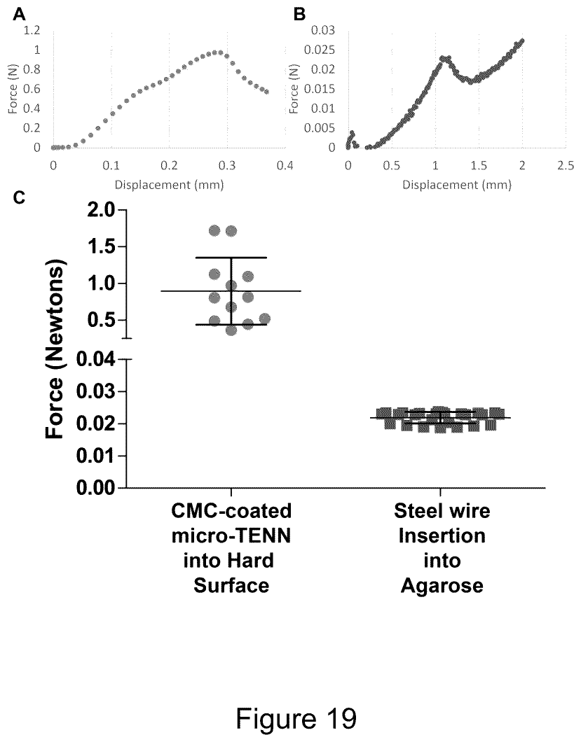

FIG. 19, comprising FIGS. 19A through 19C, depicts mechanical testing of buckling force and brain insertion force. FIG. 19A depicts a representative trace from CMC-coated Micro-TENN driven into a solid aluminum block. The force increased to a peak whereupon the Micro-TENN buckled under the force applied. The peak is the maximum force that the coated Micro-TENN could withstand before buckling (buckling force). FIG. 19B depicts a representative trace from a stainless steel wire (diameter similar to CMC-coated Micro-TENN) inserted into 0.6% agarose (brain phantom). The force increased as the wire was lowered into the agarose, and the agarose dimpled. The force built until the surface of the agarose surface was punctured by the wire, indicated by the peak around the 1 mm displacement point. This is deemed the required insertion force into the brain phantom for a material of this size and shape. Note (A) and (B) have different scales. FIG. 19C is a graph depicting forces from CMC-coated Micro-TENN buckling trials and steel wire into agarose insertion trials. Error bars are standard deviation. CMC-coated Micro-TENN were able to withstand 893+−457 mN before buckling. Similarly sized wire required 22+−2 mN of force for insertion into the brain phantom. Results indicate that the CMC-coated Micro-TENNs are much stronger than what is required for inserted into the brain.

### Example 10: Still Frames of Needle-Less CMC Micro-TENN Penetrating a Surrogate “Brain” Material

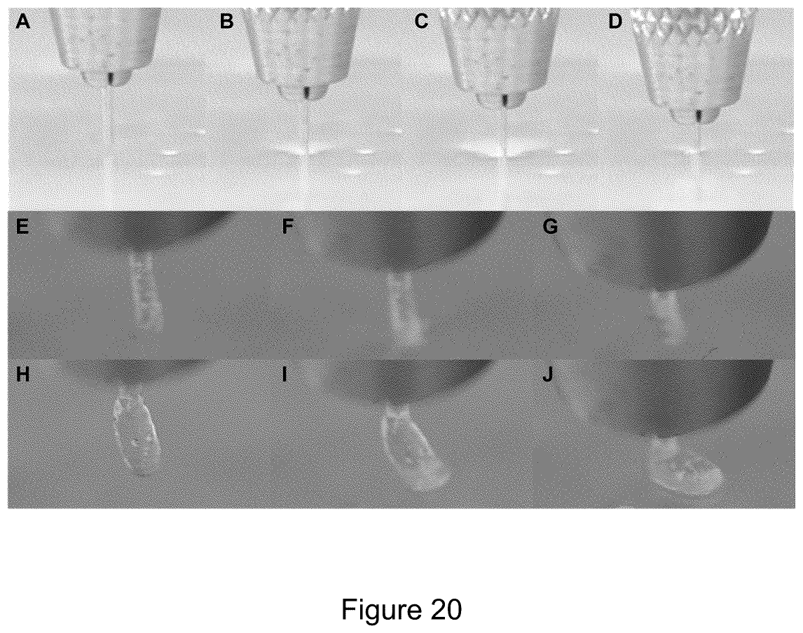

Snapshots of a movie of insertion of steel wire and CMC-coated Micro-TENN into brain phantom are depicted in FIG. 20. Steel wire (similar size to CMC-coated Micro-TENN) was implanted into 0.6% agarose (brain phantom) (FIGS. 20A-D). The frames show the several stages of insertion. The first stage shows the agarose and wire pre-movement (FIG. 20A). As the wire is advanced, the agarose dimples as force is built up (FIGS. 20B-C). The agarose continues to dimple until the surface is punctured, therefore relaxing the dimpling (FIG. 20D). CMC-coated Micro-TENN was implanted into brain phantom (FIGS. 20E-J). Similar to wire insertion, the first frame shows the Micro-TENN pre-movement (FIG. 20E). The Micro-TENN was implanted in the brain phantom (camera angle and zoom does not allow viewing of dimpling) (FIGS. 20F-G). After 30 seconds in agarose, the Micro-TENN was removed from the agarose showing hydration of CMC coating (FIG. 20H). Immediate reinsertion was attempted, but the hydrated CMC had softened. As a result, the Micro-TENN buckled during reinsertion (FIG. 20I). After the movement was completed, the Micro-TENN was completely buckled and could not be inserted into brain phantom (FIG. 20H).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1-19. (canceled)

20. A method of culturing and implanting a micro-tissue engineered neural network in a mammal to promote structural integration of the micro-tissue engineered neural network with the host brain tissue of the mammal, the method comprising:
providing an elongated tubular hydrogel construct having a first end and a second end, the construct comprising a tubular body comprising at least one biopolymer and having an inner surface defining a luminal core comprising at least one extracellular matrix protein;
positioning a plurality of neuronal progenitor cells at the first end of the construct;
culturing the plurality of neuronal progenitor cells at the first end of the construct in vitro to:
(i) mature the neuronal progenitor cells into a plurality of neurons comprising one or more differentiated neuronal subtypes selected from the group consisting of glutamatergic neurons, GABAergic neurons, and dopaminergic neurons; and
(ii) promote growth of axons of the plurality of neurons through at least a portion of the luminal core, along a length of the construct, thereby forming the micro-tissue engineered neural network comprising the construct and neurons having an engineered cytoarchitecture, such that soma of the neurons are located at the first end and the grown axons extend through the core, and run longitudinally for at least a portion of a length of construct; and
implanting the micro-tissue-engineered neural network into the mammal.

21. The method of claim 20, wherein the mammal is a rat.

22. The method of claim 20, wherein the at least one biopolymer is at least one selected from the group consisting of hyaluronan, chitosan, alginate, collagen, dextran, pectin, carrageenan, polylysine, gelatin and agarose.

23. (canceled)

24. (canceled)

25. The method of claim 20, wherein the construct has an outer diameter from about 500 μm to about 1 mm.

26. The method of claim 20, wherein the construct has an inner diameter from about 125 μm to about 500 μm.

27. The method of claim 20, wherein the length of the construct is from about 0.1 mm to about 10 cm.

28. (canceled)

29. (canceled)

30. (canceled)

31. (canceled)

32. (canceled)

33. (canceled)

34. The method of claim 20, wherein the neuronal progenitor cells are obtained from rat.

35. (canceled)

36. (canceled)

37. (canceled)

38. (canceled)

39. The method of claim 20, wherein the method restores an axonal connection in the peripheral nervous system (PNS).

40. (canceled)

41. (canceled)

42. (canceled)

43. (canceled)

44. (canceled)

45. The method of claim 20, wherein implanting the micro-tissue-engineered neural network comprises loading the micro-tissue-engineered neural network into a needle and injecting the micro-tissue-engineered neural network into a tissue of the mammal.

46. The method of claim 20, wherein implanting the micro-tissue-engineered neural network comprises directly penetrating a tissue of the mammal with the micro-tissue-engineered neural network.

47. The method of claim 20, wherein the extracellular matrix protein is selected from the group consisting of collagen, fibronectin, fibrin, hyaluronic acid, elastin, and laminin.

48. The method of claim 20, wherein the construct comprises a protective outer coating on an outer surface of the tubular body, the protective outer coating comprising carboxymethylcellulose.

49. A method of promoting neurite outgrowth in a rat, the method comprising:
providing a micro-tissue-engineered neural network, wherein:
the micro-tissue-engineered neural network comprises an elongated tubular hydrogel construct; wherein the tubular hydrogel construct having a first end and a second end, the construct comprising a tubular body comprising an agarose-extracellular matrix hydrogel, wherein the hydrogel comprises 1-4% agarose in phosphate-buffered saline; and having an inner surface defining a luminal core comprising salmon-derived fibrin matrix or a blend of collagen and laminin; and having an outer surface having a coating comprising carboxymethylcellulose;
positioning a plurality of cortical neurons isolated from rats at embryonic day 18, wherein the cortical neurons are positioned at the first end of the construct, and further wherein the plurality of cortical neurons are at a density of 56000, 72000 or 36000 cell/ml;
culturing the plurality of cortical neurons at the first end of the construct; and
implanting the micro-tissue-engineered neural network by stereotaxically injecting the micro-tissue-engineered neural network into the cortex in the rat.

50. A method of promoting neurite outgrowth in a rat, the method comprising:
providing a micro-tissue-engineered neural network, wherein:
the micro-tissue-engineered neural network comprises an elongated tubular hydrogel construct; wherein the tubular hydrogel construct comprises a first end and a second end, the construct comprising a tubular body comprising an agarose-extracellular matrix hydrogel, wherein the hydrogel comprises 1-4% agarose in phosphate-buffered saline; and an inner surface defining a luminal core comprising collagen; and having an outer surface having a coating comprising carboxymethylcellulose;
positioning a plurality of dorsal root ganglia (DRG) neurons isolated from rats at embryonic day 16, wherein the DRG neurons are positioned at the first end of the construct;
culturing the plurality of DRG neurons at the first end of the construct; and
implanting the micro-tissue-engineered neural network by stereotaxically injecting the micro-tissue-engineered neural network into the cortex in the rat.
